(19) United States
(12) Patent Application Publication
(10) Pub. No.: US 2016/0038448 A1
(43) Pub. Date: Feb. 11, 2016

(54) THE INHIBITION OF THE TOXIC EFFECTS OF ISLET AMYLOID FORMATION BY FLURBIPROFEN AND FLURBIPROFEN DERIVATIVES

(71) Applicants: Daniel RALEIGH, (US); Olesya LEVSH, (US); Ping CAO, (US); Harris NOOR, (US); Wenli MENG, (US)

(72) Inventors: Daniel RALEIGH, Stony Brook, NY (US); Olesya LEVSH, New York, NY (US); Ping CAO, Stony Brook, NY (US); Harris NOOR, Hauppauge, NY (US); Wenli MENG, Miller Place, NY (US)

(73) Assignee: The Research Foundation for the State University of New York, Albany, NY (US)

(21) Appl. No.: 14/774,042

(22) PCT Filed: Mar. 12, 2014
(86) PCT No.: PCT/US2014/025072

§ 371 Date: Sep. 9, 2015

Related U.S. Application Data
(60) Provisional application No. 61/785,024, filed on Mar. 14, 2013.

(51) Int. Cl. A61K 31/192; A61K 38/28

(52) U.S. Cl. CPC A61K 31/192; A61K 38/28

(57) ABSTRACT

The subject invention provides a method of reducing the presence of toxic intermediates of amyloidosis from islet amyloid polypeptide (IAPP) in a patient, comprising administering a compound to the patient a compound having the structure: (Formula 1) wherein R1 and R2 are each independently hydrogen, methyl, ethyl, propyl, butyl, pentyl, hexyl, or R1 and R2 are linked so as to form a cyclopropyl cyclobutyl, cyclopentyl, or cyclohexyl ring; wherein R3, R4, R5, R6 are each independently hydrogen, fluorine or chlorine; and wherein R7 is hydrogen, phenyl, fluorine, chlorine, bromine, hydroxyl, amine, carboxylic acid, C1-C6 alkyl carboxylate, amide, methyl, ethyl, butyl, pentyl, hexyl, or a pharmaceutically acceptable salt or ester thereof, so as to thereby reduce the presence of toxic intermediates of amyloidosis from islet amyloid polypeptide (IAPP) in the patient.

This application claims priority of U.S. Provisional Application No. 61/785,024, filed Mar. 14, 2013, the contents of which are hereby incorporated by reference.

Throughout this application, certain publications are referenced in parentheses. Full citations for these publications may be found immediately preceding the claims. The disclosures of these publications in their entireties are hereby incorporated by reference into this application in order to describe more fully the state of the art to which this invention relates.

The invention was made with government support under grant number GM078114 awarded by the National Institute of Health. The government has certain rights in the invention.

### BACKGROUND OF THE INVENTION

Amyloid deposits are partially ordered aggregates of normally soluble proteins. A wide range of proteins form amyloid in various patho-physiological states. The process has been implicated in more than 20 different human diseases (Chiti et al. 2006; Selkow at al. 2004).

Type 2 diabetes (T2D) has reached epidemic proportions and it is now recognized that β-cell death and β-cell dysfunction play important roles in the diseases (Ashcroft at al. 2012). A range of mechanisms contribute to β-cell loss and dysfunction in vivo, including inflammation and the deposition of amyloid in the islet of Langerhans (Donath et al. 2011; Clark at al. 1988; Lorenzo et al. 1994; Konarkowska et al. 2006). Rapid formation of amyloid also leads to islet graft failure, while its prevention has been shown to prolong graft survival and lead to improved glycemic control (Westermark et al. 2008; Potter at al. 2010). The neuropancreatic hormone amylin (also known as islet amyloid polypeptide, IAPP) is responsible for islet amyloid formation. The polypeptide normally plays a role in controlling food intake, gastric emptying and glucose homeostasis, but aggregates to form islet amyloid in T2D. Amylin is stored in the insulin secretory granule and is thus released in response to insulin secretion (Kahn et al. 1990).

Islet amyloid formation or islet amyloidosis refers to the formation of amyloid deposits in the islet of Langerhans in the pancreas. Amyloid formation in the islets of Langerhans is a characteristic feature of type 2 diabetes (T2D). Formation of islet amyloid leads to β-cell dysfunction and cell death and decline in β-cell mass (Westermark et al. 1987; Cooper et al. 1987; Westermark at al. 2011; Clark et al. 1988; Lorenzo at al. 1994; Hull et al. 2004; Montane et al. 2012). The loss of β-cell mass and function is recognized as a key event in type 2 diabetes (Ashcroft at al. 2012). Amyloid formation by islet amyloid polypeptide (IAPP, also called amylin) also leads to the failure of islet cell transplants (also called islet cell grafts) and is a major factor limiting this therapeutic avenue (Westermark at al. 2008). Prevention of islet amyloid toxicity has been shown to prolong islet cell graft survival (Potter et al. 2010). An unsolved problem in the field is determining the toxic species of amyloid formation.

There are no drugs clinically approved for inhibition of IAPP induced toxicity. A number of inhibitors of IAPP toxicity have been reported in the literature, but they have significant disadvantages. Published reports of IAPP inhibitors describe molecules which are not “drug-like”, e.g., molecules which are either peptides or complex, chemically labile polyphenols, or highly sulfonated compounds with toxicity issues (Potter et al. 2010; Abedini at al. 2007; Meng at al. 2010a, 2010b, 2010c; Hebda at al. 2009; Cao at al. 2012a; Sinha at al. 2011; Noor et al. 2012; Porat at al. 2004; Zelus at al. 2012; Cheng et al. 2012; Rigacci et al. 2010; Mishra at al. 2009; Yan et al. 2006). Polyphenols are readily modified via oxidation or hydrolysis, and the active form of these compounds is not clear. The solubility profiles of many of these inhibitors are not suitable for drugs. Further, many of these drugs have multiple effects, target other aggregation processes or act as anti-oxidants.

For example, (−)-epigallocatechin 3-gallate (EGCG), a biologically active flavanol in green tea, is probably the best studied polyphenol IAPP inhibitor, but the compound is not suitable for use as a drug (Meng et al. 2010a). The mode of action of EGCG and other polyphenols with human IAPP is not known. Other inhibitors include sulfonated triphenyl methane derivatives such as acid fuschin (Meng et al. 2010c). These compounds are inhibitors of IAPP amyloid formation and of toxicity in cell culture, although they are unlikely drug candidates since they induce irritation and do not have suitable solubility profiles. A lysine-specific molecular tweezer has been reported to have broad activity against a range of amyloid forming proteins and inhibitors hIAPP amyloid formation and toxicity, but it suffers from the same chemical issues as Acid fuschin (Sinha et al. 2011). Another class of mid-size inhibitors has been reported which target intermediates in IAPP assembly, but they too are not drug like (Hebda et al. 2009). They are highly charged, and only moderately effective in vitro.

Several rationally designed polypeptide inhibitors have been reported to inhibit human IAPP amyloid formation. However, such compounds are poor candidates for drugs because they are challenging and expensive to synthesize, and are expected not to be orally active (Abedini et al. 2007; Meng et al. 2010a; Yan et al. 2006).

Flurbiprofen [(RS)-2-(2-fluorobiphenyl-4-yl)propanoic acid] is a non-steroidal anti-inflammatory (NSAID) which has been approved for use in humans as an anti-inflammatory and pain medication. Flurbiprofen is given orally and has been shown to be safe in humans.

### SUMMARY OF THE INVENTION

The subject invention provides a method of reducing the presence of toxic intermediates of amyloidosis from islet amyloid polypeptide (IAPP) in a patient, comprising administering a compound to the patient a compound having the structure:

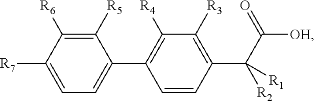

wherein R1 and R2 are each independently hydrogen, methyl, ethyl, propyl, butyl, pentyl or hexyl, or R1 and R2 are linked so as to form a cyclopropyl, cyclobutyl, cyclopentyl, or cyclohexyl ring;
wherein R3, R4, R5, R6 are each independently hydrogen, fluorine or chlorine; and
wherein R7 is hydrogen, phenyl, fluorine, chlorine, bromine, hydroxyl, amine, carboxylic acid, C1-C6 alkyl carboxylate, amide, methyl, ethyl, propyl, butyl, pentyl or hexyl, or
   a pharmaceutically acceptable salt or ester thereof,
   so as to thereby reduce the presence of toxic intermediates of amyloidosis from islet amyloid polypeptide (IAPP) in the patient. The subject invention provides a method of reducing toxicity of amyloidosis from islet amyloid polypeptide (IAPP) in a patient, comprising administering to the patient a compound having the structure:

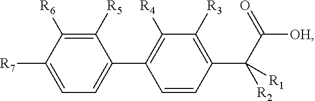

wherein R1 and R2 are each independently hydrogen, methyl, ethyl, propyl, butyl, pentyl or hexyl, or R1 and R2 are linked so as to form a cyclopropyl, cyclobutyl, cyclopentyl, or cyclohexyl ring;
wherein R3, R4, R5, R6 are each independently hydrogen, fluorine or chlorine; and
wherein R7 is hydrogen, phenyl, fluorine, chlorine, bromine, hydroxyl, amine, carboxylic acid, C1-C6 alkyl carboxylate, amide, methyl, ethyl, propyl, butyl, pentyl or hexyl, or
   a pharmaceutically acceptable salt or ester thereof,
   so as to thereby reduce the toxicity of amyloidosis from islet amyloid polypeptide (IAPP) in the patient.

The subject invention provides a method of enhancing the survival of islet cell transplants in a patient, comprising administering to the patient a compound having the structure:

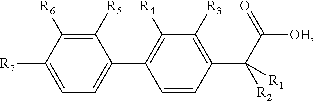

wherein R1 and R2 are each independently hydrogen, methyl, ethyl, propyl, butyl, pentyl or hexyl, or R1 and R2 are linked so as to form a cyclopropyl, cyclobutyl, cyclopentyl, or cyclohexyl ring;
wherein R3, R4, R5, R6 are each independently hydrogen, fluorine or chlorine; and
wherein R7 is hydrogen, phenyl, fluorine, chlorine, bromine, hydroxyl, amine, carboxylic acid, C1-C6 alkyl carboxylate, amide, methyl, ethyl, propyl, butyl, pentyl or hexyl, or
   a pharmaceutically acceptable salt or ester thereof,
   so as to thereby enhance the survival of islet cell transplants.

### BRIEF DESCRIPTION OF THE FIGURES

FIG. 1. S-Flurbiprofen accelerates amyloid formation by human IAPP in a dose dependent manner. (A) Amyloid formation in the presence and absence of flurbiprofen monitored by fluorescence detected thioflavin-T assays. (a): human IAPP; (b): IAPP:flurbiprofen at a 1:1 ratio; (c): IAPP:flurbiprofen at a 1:5 ratio; (d): IAPP:flurbiprofen at a 1:10 ratio; (e): IAPP:flurbiprofen at a 1:20 ratio. (B) A plot of T₅₀, the time required for 50% of the signal change in a thioflavin-T experiment vs. flurbiprofen concentration. (C) TEM images collected at the end of the reactions shown in panel-A. The same letters refer to the same mixtures in all panels. Scale bars represent 100 nm.

FIG. 2. A thioflavin-T fluorescence detected kinetic assay shows that both R and S flurbiprofen and the racemic mixture are equally effective. Experiments were performed at a Peptide:Compound ratio of 1:20.

FIG. 3. Flurbiprofen induced aggregates are rich in β-structure and seed amyloid formation by human IAPP. (A) IR spectra of the amyloid fibers formed at the end of the reaction depicted by the blue curve in FIG. 1A (IAPP:flurbiprofen=1:20). The top spectrum is a one dimension IR spectrum. The bottom spectrum is a two dimensional IR spectrum. Both spectra show that significant amounts of beta sheet structure are formed, as expected for an amyloid fiber. (B) Seeding experiments: Seeding refers to the addition of a small amyloid of pre-formed amyloid fibers to an initially unaggerated solution of polypeptide. Addition of the amyloid fibers leads to a by passing of the lag phase. Seeding is usually specific in the sense that amyloid formed by one protein usually does not see amyloid formation by a different protein. (a): unseeded human IAPP. (b): human IAPP seeded by human IAPP fibers. (c): human IAPP seeded by fibers formed by the 1:20 IAPP:flurbiprofen mixture. The insert shows a TEM imagine of the fibers formed in the IAPP:flurbiprofen 1:20 seeded reaction (scale bar represents 100 nm). The highly efficient seeding indicates that the structure of the amyloid fibers formed in the presence of the compound is very similar to the structure of the amyloid formed in its absence.

FIG. 4. A chart showing a thioflavin-T fluorescence detected kinetic assay is showing that 2-(2-fluoro-4′-hydroxybiphenyl-4-yl) propanoic acid (OH-Flurbiprofen) accelerates amyloid formation by human islet amyloid polypeptide. The chart includes election microcopy images recorded of samples at the end of the assay.

FIG. 5. Flurbiprofen protects β-cells against IAPP induced toxicity. (A) A thioflavin-T monitored time course of amyloid formation in the presence and absence of flurbiprofen. Aliquots were removed at various time points as showed in (A) and applied to rat INS-1 beta-cells; (a) thirty minutes, (b) 12 hours, (c) 48 hours (B) Cell viability as judged by AlamarBlue assays. 20 mM Tris-HCl buffer with 0.25% DMSO was used as the control.

FIG. 6. Flurbiprofen is monomeric. (A) Aromatic region of the NMR spectrum of flurbiprofen collected on 32 and 640 micro molar samples. The peak positions and line widths are independent of concentration. (B) PFG-NMR diffusion experiments. The experiment allows measurement of the hydrodynamic radius of a compound. The hydrodynamic radius is very sensitive to aggregation and increases dramatically if a molecules aggregates. The decay curve for the methyl resonance of 640 micromolar flurbiprofen is shown. The best fit to the data yields a hydrodynamic radius of 3.9 Å. The insert is the structure of flurbiprofen. The calculated value of Rₕ is 3.4 Å. The small difference between experiment and calculated values are not significant.

FIG. 7. Additional data showing Flurbiprofen is monomeric and does not form micelles. Flurbiprofen does not induce the non-amyloidogenic point mutant I26P-IAPP to form amyloid fibers, but 100% anionic vesicles do. (A) Sequence of I26P-IAPP. (B) Amyloid formation monitored by fluorescence detected thioflavin-T assays. (d); I26P-IAPP in buffer. (e); 1:20 mixture of I26P flurbiprofen. (c); I26P-IAPP in the presence of 100% DOPG vesicles. TEM images collected at the time points indicted by the colored triangles. (C) Image of I26P in the presence of 100% DOPG vesicles. (0) Image of I26P-IAPP in buffer. (E) Image of 1:20 mixture of I26P-IAPP with flurbiprofen. Scale bars represent 100 nm. (F) Anioinc vesicles accelerate amyloid formation by IAPP. Thioflavin-T assays of amyloid formation are shown for human IAPP in the presence and absence of vesicles composed of anionic lipids. The data shows that materials which form anionic vesicles or micelles can accelerate amyloid formation by human IAPP.

FIG. 8. Flurbiprofen does not accelerate amyloid formation by the Aβ₁₋₄₀ peptide. This experiment shows that flurbiprofen does not accelerate amyloid formation by all proteins. (A) Amyloid formation monitored by fluorescence detected thioflavin-T assays: (c) Aβ₁₋₄₀, (b) 1:20 mixture of Aβ₁₋₄₀ and flurbiprofen (b); (B) TEM image of Aβ₁₋₄₀ collected at the end of the reaction; and (C) TEM image of 1:20 mixture of Aβ₁₋₄₀ and flurbiprofen collected at the end of the reaction. Scale bars represent 100 nm.

FIG. 9. Flurbiprofen accelerates amyloid formation by the 3XL-IAPP mutant and by H18Q-IAPP mutant, indicating that neither aromatic residues nor His-18 are critical for the acceleration effects. (A) Sequence of the mutants. Amyloid formation monitored by fluorescence detected thioflavin-T assays (B) (a): 3X-IAPP; (b): 1:20 mixture of 3XL-IAPP with flurbiprofen. (C) (c): H18Q-IAPP; (d): 1:20 mixture of H18Q-IAPP with flurbiprofen. TEM images were collected at the end of the reactions, at the time points indicted by the colored triangles. Scale bars represent 100 nm.

FIG. 10. Other non-steroidal anti-inflammatory drugs do not accelerate human IAPP amyloid formation as efficiently as flurbiprofen does. (A) Structure of aspirin; (B) Structure of naproxen; (C) Amyloid formation in the absence or presence of the small molecules monitored by fluorescence detected thioflavin-T assays. Chart shows: human IAPP; 1:20 mixture of human IAPP with aspirin; 1:20 mixture of human IAPP with Naproxen; and 1:20 mixture of human IAPP with flurbiprofen.

FIG. 11. Aspirin and Naproxen show no inhibition of amyloid. Aspirin was tested in a 20 to 1 ration of Aspirin to Human IAPP. Naproxen was tested in a 20 to 1 ration of Naproxen to Human IAPP.

FIG. 12. Primary sequence of human amylin and the structure of aspirin and Ketoprofen. Amylin contains a disulfide bridge between residues 2 and 7, and the C-terminus is amidated.

FIG. 13. Aspirin does not inhibit amyloid formation by human amylin. (A) thioflavin-T fluorescence assays of the time course of amyloid formation in the absence (black) and in the presence (red) of 20-fold excess of aspirin. (B) TEM image of the sample of amylin without aspirin recorded at the end of the kinetic experiment. (C) TEM image of the sample of amylin with 20-fold excess aspirin recorded at the end of the kinetic experiment. Scale bars represent 100 nm. Experiments were conducted at pH 7.4 25° C. in 20 mM Tris HCl, 0.25% DMSO (V/V) in the absence of any fluorinated alcohol co-solvent. The concentration of amylin was 16 μM and the concentration of aspirin was 320 μM.

FIG. 14. Aspirin does not inhibit amyloid formation by human amylin. (A) Right angle light scattering assays of the time course of amyloid formation in the absence and in the presence of 20-fold excess of aspirin. No thioflavin-T was added to either sample (B) TEM image of the sample of amylin without aspirin recorded at the end of the kinetic experiment. (C) TEM image of the sample of amylin with 20 fold excess aspirin recorded at the end of the kinetic experiment. Scale bars represent 100 nm. Experiments were conducted at pH 7.4 25° C. in 20 mM Tris HCl, 0.25% DMSO (V/V) in the absence of any fluorinated alcohol co-solvent. The concentration of amylin was 16 μm and the concentration of aspirin was 320 μM.

FIG. 15. Aspirin does not disaggregate pre-formed human amylin amyloid fibrils. (A) thioflavin-T fluorescence assays of the time course of amyloid formation. A 20-fold excess of aspirin was added at the time point indicated by the red arrow. (B) TEM image of the sample just before the addition of 20-fold excess aspirin, black star (C) TEM image of the sample just after the addition of 20-fold excess aspirin, green star (D) TEM image of the sample 48 hours after the addition of 20-fold excess aspirin, blue star. Scale bars represent 100 nm. Experiments were conducted at pH 7.4 25° C. in 20 mM Tris HCl, 0.25% DMSO (V/V) in the absence of any fluorinated alcohol co-solvent. The concentration of amylin was 16 μM and the concentration of aspirin was 320 μM.

FIG. 16: Ketoprofen does not inhibit amyloid formation by amylin. TEM images of samples recorded after incubating amylin with varying amount of Ketoprofen for 42 hours. Scale bars represent 100 nm. (A) Amylin alone. (B) Mixture of amylin with 20-fold excess of Ketoprofen. (C) Mixture of amylin with 10-fold excess of Ketoprofen. (D) Mixture of amylin with 5-fold excess of Ketoprofen. (E) Mixture of amylin with 2-fold excess of Ketoprofen. (F) Mixture of amylin with equimolar amount of Ketoprofen. Experiments were conducted at pH 7.4 25° C. in 20 mM Tris HCl, 0.25% DMSO (V/V) in the absence of any fluorinated alcohol co-solvent. The concentration of amylin was 16 μM.

### DETAILED DESCRIPTION OF THE INVENTION

The subject invention provides a method of reducing the presence of toxic intermediates of amyloidosis from islet amyloid polypeptide (IAPP) in a patient, comprising administering a compound to the patient a compound having the structure:

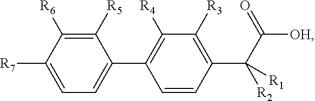

wherein R1 and R2 are each independently hydrogen, methyl, ethyl, propyl, butyl, pentyl or hexyl, or R1 and R2 are linked so as to form a cyclopropyl, cyclobutyl, cyclopentyl, or cyclohexyl ring;
wherein R3, R4, R5, R6 are each independently hydrogen, fluorine or chlorine; and
wherein R7 is hydrogen, phenyl, fluorine, chlorine, bromine, hydroxyl, amine, carboxylic acid, C1-C6 alkyl carboxylate, amide, methyl, ethyl, propyl, butyl, pentyl or hexyl, or
   a pharmaceutically acceptable salt or ester thereof,
   so as to thereby reduce the presence of toxic intermediates of amyloidosis from islet amyloid polypeptide (IAPP) in the patient.

The subject invention provides a method of reducing toxicity of amyloidosis from islet amyloid polypeptide (IAPP) in a patient, comprising administering to the patient a compound having the structure:

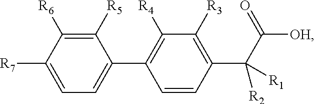

wherein R1 and R2 are each independently hydrogen, methyl, ethyl, propyl, butyl, pentyl or hexyl, or R1 and R2 are linked so as to form a cyclopropyl, cyclobutyl, cyclopentyl, or cyclohexyl ring;
wherein R3, R4, R5, R6 are each independently hydrogen, fluorine or chlorine; and
wherein R7 is hydrogen, phenyl, fluorine, chlorine, bromine, hydroxyl, amine, carboxylic acid, C1-C6 alkyl carboxylate, amide, methyl, ethyl, propyl, butyl, pentyl or hexyl, or
   a pharmaceutically acceptable salt or ester thereof,
   so as to thereby reduce the toxicity of amyloidosis from islet amyloid polypeptide (IAPP) in the patient.

In some embodiments of the method, the toxicity is to islet cells.

In some embodiments of the method, the toxicity is to β-cells.

In some embodiments of the method, the toxicity is to insulin-producing cells.

In some embodiments of the method, the toxicity causes β-cell dysfunction, β-cell cell death or decline in β-cell mass.

In some embodiments of the method, the toxicity causes progression of type-2 diabetes in the patient.

In some embodiments of the method, the toxicity causes insulin resistance, impaired glucose tolerance or insulin deficiency in the patient.

The subject invention provides a method of enhancing the survival of islet cell transplants in a patient, comprising administering to the patient a compound having the structure:

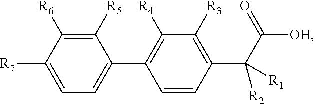

wherein R1 and R2 are each independently hydrogen, methyl, ethyl, propyl, butyl, pentyl or hexyl, or R1 and R2 are linked so as to form a cyclopropyl, cyclobutyl, cyclopentyl, or cyclohexyl ring;
wherein R3, R4, R5, R6 are each independently hydrogen, fluorine or chlorine; and
wherein R7 is hydrogen, phenyl, fluorine, chlorine, bromine, hydroxyl, amine, carboxylic acid, C1-C6 alkyl carboxylate, amide, methyl, ethyl, propyl, butyl, pentyl or hexyl, or
   a pharmaceutically acceptable salt or ester thereof,
   so as to thereby enhance the survival of islet cell transplants.

In some embodiments of the method, the compound is administered to the patient before the patient receives islet cell transplants.

In some embodiments of the method, the compound is administered to the patient after the patient receives islet cell transplants.

In some embodiments of the method, the compound is administered to the patient concurrently with islet cell transplants.

In some embodiments of the method, the administration prevents the failure of islet cell transplants.

In some embodiments of the method, the islet cell transplants are human islet cells.

In some embodiments of the method, the compound has the structure:

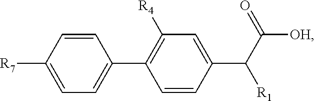

wherein
R1 is hydrogen or methyl;
R4 hydrogen or fluorine; and
R7 is hydrogen or hydroxyl, or
   a pharmaceutically acceptable salt or ester thereof.

In some embodiments of the method, the compound has the structure:

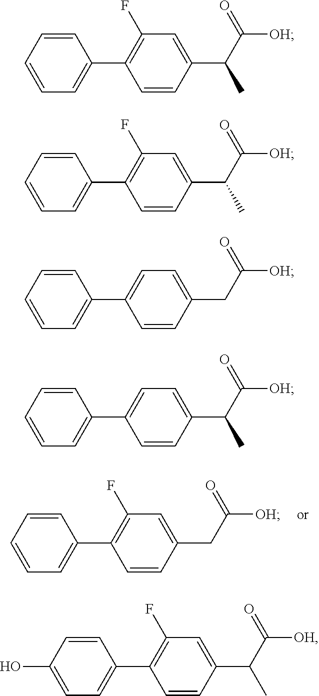

or
a pharmaceutically acceptable salt or ester thereof.

In some embodiments of the method, the compound has the structure:

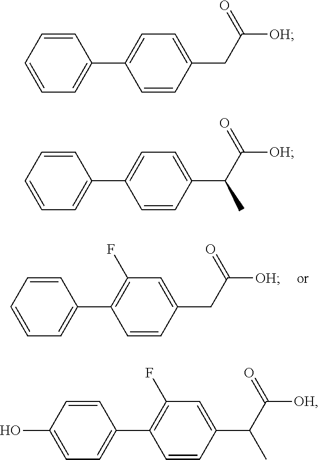

or
a pharmaceutically acceptable salt or ester thereof.

In some embodiments of the method, the compound has the structure:

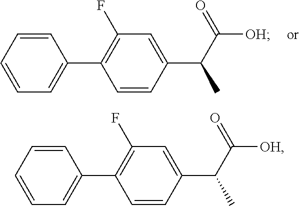

or
a pharmaceutically acceptable salt or ester thereof.

In some embodiments of the method, the structure:

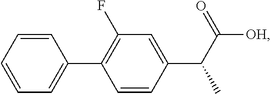 [SMILES: C[C@@H](C(=O)O)c1ccc(-c2ccccc2)c(F)c1]

or
a pharmaceutically acceptable salt or ester thereof.

In some embodiments of the method, the structure:

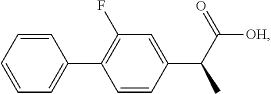 [SMILES: C[C@H](C(=O)O)c1ccc(-c2ccccc2)c(F)c1]

or
a pharmaceutically acceptable salt or ester thereof.

In some embodiments of the method, the patient is at risk for amyloidosis from islet amyloid polypeptide (IAPP).

In some embodiments of the method, the compound is administered to the patient exhibiting amyloidosis from islet amyloid polypeptide (IAPP).

In some embodiments of the method, the amyloidosis from islet amyloid polypeptide (IAPP) is in the pancreas.

In some embodiments of the method, the amyloidosis from islet amyloid polypeptide (IAPP) is in the islet of Langerhans.

In some embodiments of the method, the patient is a human.

In some embodiments of the method, the human exhibits a mutant form of islet amyloid polypeptide (IAPP).

In some embodiments of the method, the patient is a domesticated animal.

In some embodiments of the method, the domesticated animal is a cat or ferret.

In some embodiments of the method, the compound accelerates amyloidosis from islet amyloid polypeptide (IAPP).

In some embodiments of the method, the compound is administered orally, intravenously, intranasally, transdermally, intraperitoneal, subcutaneously, intramuscularly or via direct injection to the pancreas.

In some embodiments of the method, the compound is administered orally.

In some embodiments of the method, the compound is administered intravenously, intranasally, transdermally, intraperitoneal, subcutaneously, intramuscularly or via direct injection to the pancreas.

In some embodiments of the method, the compound is administered with a pharmaceutically acceptable carrier.

In some embodiments of the method, the compound is co-administered with insulin.

Except where otherwise specified, when the structure of a compound of this invention includes an asymmetric carbon atom, it is understood that the compound occurs as a racemate, racemic mixture, and isolated single enantiomer. All such isomeric forms of these compounds are expressly included in this invention. Except where otherwise specified, each stereogenic carbon may be of the R or S configuration. It is to be understood accordingly that the isomers arising from such asymmetry (e.g., all enantiomers and diastereomers) are included within the scope of this invention, unless indicated otherwise. Such isomers can be obtained in substantially pure form by classical separation techniques and by stereochemically controlled synthesis, such as those described in “Enantiomers, Racemates and Resolutions” by J. Jacques, A. Collet and S. Wilen, Pub. John Wiley & Sons, NY, 1981. For example, the resolution may be carried out by preparative chromatography on a chiral column.

The subject invention is also intended to include all isotopes of atoms occurring on the compounds disclosed herein. Isotopes include those atoms having the same atomic number but different mass numbers. By way of general example and without limitation, isotopes of hydrogen include tritium and deuterium. Isotopes of carbon include C-13 and C-14. Isotopes of nitrogen include N-15. Isotopes of oxygen include 0-17 and 0-18. Isotopes of fluorine include F-18.

It will be noted that any notation of a carbon in structures throughout this application, when used without further notation, are intended to represent all isotopes of carbon, such as ¹²C, ¹³C, or ¹⁴C. Furthermore, any compounds containing ¹³C or ¹⁴C may specifically have the structure of any of the compounds disclosed herein.

It will also be noted that any notation of a hydrogen in structures throughout this application, when used without further notation, are intended to represent all isotopes of hydrogen, such as ¹H, ²H, or ³H. Furthermore, any compounds containing ²H or ³H may specifically have the structure of any of the compounds disclosed herein.

It will also be noted that any notation of a nitrogen in structures throughout this application, when used without further notation, are intended to represent all isotopes of nitrogen, such as ¹⁵N. Furthermore, any compounds containing ¹⁵N may specifically have the structure of any of the compounds disclosed herein.

It will also be noted that any notation of a oxygen in structures throughout this application, when used without further notation, are intended to represent all isotopes of oxygen, such as ¹⁷O or ¹⁸O. Furthermore, any compounds containing ¹⁷O or ¹⁸O may specifically have the structure of any of the compounds disclosed herein.

It will also be noted that any notation of a fluorine in structures throughout this application, when used without further notation, are intended to represent all isotopes of fluorine, such as ¹⁸F. Furthermore, any compounds containing ¹⁸F may specifically have the structure of any of the compounds disclosed herein.

Isotopically-labeled compounds can generally be prepared by conventional techniques known to those skilled in the art using appropriate isotopically-labeled reagents in place of the non-labeled reagents employed.

The term “substitution”, “substituted” and “substituent” refers to a functional group as described above in which one or more bonds to a hydrogen atom contained therein are replaced by a bond to non-hydrogen or non-carbon atoms, provided that normal valencies are maintained and that the substitution results in a stable compound. Substituted groups also include groups in which one or more bonds to a carbon(s) or hydrogen(s) atom are replaced by one or more bonds, including double or triple bonds, to a heteroatom. Examples of substituent groups include the functional groups described above, and halogens (i.e., F, Cl, Br, and I); alkyl groups, such as methyl, ethyl, n-propyl, isopropryl, n-butyl, tert-butyl, and trifluoromethyl; hydroxyl; alkoxy groups, such as methoxy, ethoxy, n-propoxy, and isopropoxy; aryloxy groups, such as phenoxy; arylalkyloxy, such as benzyloxy (phenylmethoxy) and p-trifluoromethylbenzyloxy(4-trifluoromethylphenylmethoxy); heteroaryloxy groups; sulfonyl groups, such as trifluoromethanesulfonyl, methanesulfonyl, and p-toluenesulfonyl; nitro, nitrosyl; mercapto; sulfanyl groups, such as methylsulfanyl, ethylsulfanyl and propylsulfanyl; cyano; amino groups, such as amino, methylamino, dimethylamino, ethylamino, and diethylamino; and carboxyl. Where multiple substituent moieties are disclosed or claimed, the substituted compound can be independently substituted by one or more of the disclosed or claimed substituent moieties, singly or plurally. By independently substituted, it is meant that the (two or more) substituents can be the same or different.

In the compounds for the method of the present invention, the substituents may be substituted or unsubstituted, unless specifically defined otherwise.

In the compounds for the method of the present invention, alkyl, heteroalkyl, monocycle, bicycle, aryl, heteroaryl and heterocycle groups can be further substituted by replacing one or more hydrogen atoms with alternative non-hydrogen groups. These include, but are not limited to, halo, hydroxy, mercapto, amino, carboxy, cyano and carbamoyl.

It is understood that substituents and substitution patterns on the compounds for the method of the present invention can be selected by one of ordinary skill in the art to provide compounds that are chemically stable and that can be readily synthesized by techniques known in the art from readily available starting materials. If a substituent is itself substituted with more than one group, it is understood that these multiple groups may be on the same carbon or on different carbons, so long as a stable structure results.

In choosing the compounds for the method of the present invention, one of ordinary skill in the art will recognize that the various substituents, i.e. R₁, R₂, etc. are to be chosen in conformity with well-known principles of chemical structure connectivity.

As used herein, “alkyl” includes cyclic, branched and straight-chain saturated aliphatic hydrocarbons, and unless otherwise specified contains one to ten carbons. Examples include, but are not limited to, methyl, ethyl, n-propyl, isopropyl, n-butyl, t-butyl, pentyl, hexyl, and octyl. Alkyl groups can be unsubstituted or substituted with one or more substituents, including but not limited to halogen, alkoxy, alkylthio, trifluoromethyl, difluoromethyl, methoxy, and hydroxyl. “C1-C6 alkyl” includes any linear or branched alkyl group having one to six carbons. The alkyl group may be substituted or unsubstituted, including substituents which add additional carbons.

As used herein, “halogen” is intended to include fluorine, chlorine, bromine and iodide.

As used herein, “aryl” is intended to “carbocyclic aryl” and “heterocyclic aryl”.

As used herein, “carbocyclic aryl” is intended to mean any stable monocyclic, bicyclic or polycyclic carbon ring of up to 10 atoms in each ring, wherein at least one ring is aromatic, and may be unsubstituted or substituted. Examples of such carbocyclic aryl elements include but are not limited to: phenyl, p-toluenyl(4-methylphenyl), naphthyl, tetrahydro-naphthyl, indanyl, phenanthryl, anthryl or acenaphthyl. In cases where the carbocyclic aryl substituent is bicyclic and one ring is non-aromatic, it is understood that attachment is via the aromatic ring.

As used herein, “heterocyclic aryl”, “heteroaryl” or “heterocycle”, is intended to mean a stable monocyclic, bicyclic or polycyclic ring of up to 10 atoms in each ring, wherein at least one ring is aromatic and contains from 1 to 4 heteroatoms selected from the group consisting of O, N and S. Bicyclic aromatic heteroaryl groups include but are not limited to phenyl, pyridine, pyrimidine or pyridizine rings that are (a) fused to a 6-membered aromatic (unsaturated) heterocyclic ring having one nitrogen atom; (b) fused to a 5- or 6-membered aromatic (unsaturated) heterocyclic ring having two nitrogen atoms; (c) fused to a 5-membered aromatic (unsaturated) heterocyclic ring having one nitrogen atom together with either one oxygen or one sulfur atom; or (d) fused to a 5-membered aromatic (unsaturated) heterocyclic ring having one heteroatom selected from O, N or S. Heteroaryl groups within the scope of this definition include but are not limited to: benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrazolyl, tetrazolopyridyl, thiadiazolyl, thiazolyl, thienyl, triazolyl, azetidinyl, aziridinyl, 1,4-dioxanyl, hexahydroazepinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, tetrahydrothienyl, acridinyl, carbazolyl, cinnolinyl, quinoxalinyl, pyrrazolyl, indolyl, benzotriazolyl, benzothiazolyl, benzoxazolyl, isoxazolyl, isothiazolyl, furanyl, thienyl, benzothienyl, benzofuranyl, quinolinyl, isoquinolinyl, oxazolyl, isoxazolyl, indolyl, pyrazinyl, pyridazinyl, pyridinyl, pyrimidinyl, pyrrolyl, tetra-hydroquinoline, in cases where the heteroaryl substituent is bicyclic and one ring is non-aromatic or contains no heteroatoms, it is understood that attachment is via the aromatic ring or via the heteroatom containing ring, respectively. If the heteroaryl contains nitrogen atoms, it is understood that the corresponding N-oxides thereof are also encompassed by this definition.

The compounds for the method of the present invention may be prepared by techniques well know in organic synthesis and familiar to a practitioner ordinarily skilled in the art. However, these may not be the only means by which to synthesize or obtain the desired compounds.

The compounds for the method of the present invention may be prepared by techniques described in Vogel's Textbook of Practical Organic Chemistry, A. I. Vogel, A. R. Tatchell, B. S. Furnis, A. J. Hannaford, P. W. G. Smith, (Prentice Hall) 5th Edition (1996), March's Advanced Organic Chemistry: Reactions, Mechanisms, and Structure, Michael B. Smith, Jerry March, (Wiley-Interscience) 5^{th} Edition (2007), and references therein, which are incorporated by reference herein. However, these may not be the only means by which to synthesize or obtain the desired compounds.

Another aspect of the invention is a pharmaceutical composition comprising the compound for the method of the present invention.

Another aspect of the invention comprises a compound used in the method of the present invention as a pharmaceutical composition.

As used herein, the term “pharmaceutically active agent” means any substance or compound suitable for administration to a subject and furnishes biological activity or other direct effect in the treatment, cure, mitigation, diagnosis, or prevention of disease, or affects the structure or any function of the subject. Pharmaceutically active agents include, but are not limited to, substances and compounds described in the Physicians' Desk Reference (PDR Network, LLC; 64th edition; Nov. 15, 2009) and “Approved Drug Products with Therapeutic Equivalence Evaluations” (U.S. Department Of Health And Human Services, 30^{th} edition, 2010), which are hereby incorporated by reference. Pharmaceutically active agents which have pendant carboxylic acid groups may be modified in accordance with the present invention using standard esterification reactions and methods readily available and known to those having ordinary skill in the art of chemical synthesis. Where a pharmaceutically active agent does not possess a carboxylic acid group, the ordinarily skilled artisan will be able to design and incorporate a carboxylic acid group into the pharmaceutically active agent where esterification may subsequently be carried out so long as the modification does not interfere with the pharmaceutically active agent's biological activity or effect.

The compounds for the method of the present invention may be in a salt form. As used herein, a “salt” is a salt of the instant compounds which has been modified by making acid or base salts of the compounds. In the case of compounds used to treat an infection or disease caused by a pathogen, the salt is pharmaceutically acceptable. Examples of pharmaceutically acceptable salts include, but are not limited to, mineral or organic acid salts of basic residues such as amines; alkali or organic salts of acidic residues such as phenols. The salts can be made using an organic or inorganic acid. Such acid salts are chlorides, bromides, sulfates, nitrates, phosphates, sulfonates, formates, tartrates, maleates, malates, citrates, benzoates, salicylates, ascorbates, and the like. Phenolate salts are the alkaline earth metal salts, sodium, potassium or lithium. The term “pharmaceutically acceptable salt” in this respect, refers to the relatively non-toxic, inorganic and organic acid or base addition salts of compounds of the present invention. These salts can be prepared in situ during the final isolation and purification of the compounds for the method of the invention, or by separately reacting a purified compound of the invention in its free base or free acid form with a suitable organic or inorganic acid or base, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, e.g., Berge et al. (1977) “Pharmaceutical Salts”, J. Pharm. Sci. 66:1-19).

As used herein, “treating” means preventing, slowing, halting, or reversing the progression of a disease or infection. Treating may also mean improving one or more symptoms of a disease or infection. For example, treating type 2 diabetes would include, but is not limited to, preventing, slowing, halting, or reversing progression of the disease in an individual from healthy to prediabetic or from prediabetic to diabetic.

The compounds for the method of the present invention may be administered in various forms, including those detailed herein. The treatment with the compound may be a component of a combination therapy or an adjunct therapy, i.e. the subject or patient in need of the drug is treated or given another drug for the disease in conjunction with one or more of the instant compounds. This combination therapy can be sequential therapy where the patient is treated first with one drug and then the other or the two drugs are given simultaneously. These can be administered independently by the same route or by two or more different routes of administration depending on the dosage forms employed.

As used herein, a “pharmaceutically acceptable carrier” is a pharmaceutically acceptable solvent, suspending agent or vehicle, for delivering the instant compounds to the animal or human. The carrier may be liquid or solid and is selected with the planned manner of administration in mind. Liposomes are also a pharmaceutically acceptable carrier.

The dosage of the compounds for the method administered in treatment will vary depending upon factors such as the pharmacodynamic characteristics of a specific chemotherapeutic agent and its mode and route of administration; the age, sex, metabolic rate, absorptive efficiency, health and weight of the recipient; the nature and extent of the symptoms; the kind of concurrent treatment being administered; the frequency of treatment with; and the desired therapeutic effect.

A dosage unit of the compounds for the method of the present invention may comprise a single compound or mixtures thereof with additional antibacterial agents. The compounds for the method can be administered in oral dosage forms as tablets, capsules, pills, powders, granules, elixirs, tinctures, suspensions, syrups, and emulsions. The compounds may also be administered in intravenous (bolus or infusion), intraperitoneal, subcutaneous, or intramuscular form, or introduced directly, e.g. by injection, topical application, or other methods, into or onto a site of infection, all using dosage forms well known to those of ordinary skill in the pharmaceutical arts.

The compounds of the present invention can be administered in admixture with suitable pharmaceutical diluents, extenders, excipients, or carriers (collectively referred to herein as a pharmaceutically acceptable carrier) suitably selected with respect to the intended form of administration and as consistent with conventional pharmaceutical practices. The unit will be in a form suitable for oral, rectal, topical, intravenous or direct injection or parenteral administration. The compounds can be administered alone or mixed with a pharmaceutically acceptable carrier. This carrier can be a solid or liquid, and the type of carrier is generally chosen based on the type of administration being used. The active agent can be co-administered in the form of a tablet or capsule, liposome, as an agglomerated powder or in a liquid form. Examples of suitable solid carriers include lactose, sucrose, gelatin and agar. Capsule or tablets can be easily formulated and can be made easy to swallow or chew; other solid forms include granules, and bulk powders. Tablets may contain suitable binders, lubricants, diluents, disintegrating agents, coloring agents, flavoring agents, flow-inducing agents, and melting agents. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents. Oral dosage forms optionally contain flavorants and coloring agents. Parenteral and intravenous forms may also include minerals and other materials to make them compatible with the type of injection or delivery system chosen.

Techniques and compositions for making dosage forms useful in the present invention are described in the following references: 7 Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol. 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modem Pharmaceutics Drugs and the Pharmaceutical Sciences, Vol 40 (Gilbert S. Banker, Christopher T. Rhodes, Eds.). All of the aforementioned publications are incorporated by reference herein.

Tablets may contain suitable binders, lubricants, disintegrating agents, coloring agents, flavoring agents, flow-inducing agents, and melting agents. For instance, for oral administration in the dosage unit form of a tablet or capsule, the active drug component can be combined with an oral, non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol and the like. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

The compounds of the present invention may also be administered in the form of liposome delivery systems, such as small unilamellar vesicles, large unilamallar vesicles, and multilamellar vesicles. Liposomes can be formed from a variety of phospholipids, such as cholesterol, stearylamine, or phosphatidylcholines. The compounds may be administered as components of tissue-targeted emulsions.

The compounds of the present invention may also be coupled to soluble polymers as targetable drug carriers or as a prodrug. Such polymers include polyvinylpyrrolidone, pyran copolymer, polyhydroxylpropylmethacrylamide-phenol, polyhydroxy-ethylasparta-midephenol, or polyethyleneoxide-polylysine substituted with palmitoyl residues. Furthermore, the compounds may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

Gelatin capsules may contain the active ingredient compounds and powdered carriers, such as lactose, starch, cellulose derivatives, magnesium stearate, stearic acid, and the like. Similar diluents can be used to make compressed tablets. Both tablets and capsules can be manufactured as immediate release products or as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract.

For oral administration in liquid dosage form, the oral drug components are combined with any oral, non-toxic, pharmaceutically acceptable inert carrier such as ethanol, glycerol, water, and the like. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents.

Liquid dosage forms for oral administration can contain coloring and flavoring to increase patient acceptance. In general, water, a suitable oil, saline, aqueous dextrose (glucose), and related sugar solutions and glycols such as propylene glycol or polyethylene glycols are suitable carriers for parenteral solutions. Solutions for parenteral administration preferably contain a water soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol. Suitable pharmaceutical carriers are described in Remington's Pharmaceutical Sciences, Mack Publishing Company, a standard reference text in this field.

The compounds of the present invention may also be administered in intranasal form via use of suitable intranasal vehicles, or via transdermal routes, using those forms of transdermal skin patches well known to those of ordinary skill in that art. To be administered in the form of a transdermal delivery system, the dosage administration will generally be continuous rather than intermittent throughout the dosage regimen.

Parenteral and intravenous forms may also include minerals and other materials to make them compatible with the type of injection or delivery system chosen.

Examples of patients which are at risk for amyloid formation include, but are not limited to, pre-diabetic patients, diabetic patients, patients with a family history of diabetes, patients with a mutant form of IAPP, and patients having lifestyle risk factors such as obesity, lack of physical activity, poor diet, stress and urbanization. “At risk” patients may fall into one or several of these categories.

As used herein, “insulin resistance”, is the inability of cells to respond adequately to normal levels of insulin, and occurs primarily within the muscles, liver, and fat tissue.

As used herein, “impaired glucose tolerance” is a pre-diabetic state of hyperglycemia that may be associated with insulin resistance and increased risk of cardiovascular pathology.

As used herein, a “β-cell” or “beta cell” is a type of cell commonly found in the pancreas in the islet of Langerhans. The primary function of β-cells is to store and release insulin. Another function of β-cells is to secret C-peptide, which prevents neuropathy and other vascular deterioration, and IAPP, which slows the rate of glucose entering the bloodstream, β-cells dysfunction involves abnormal or decreased ability of β-cells to perform any of the above-mentioned functions.

As used herein, to “enhance the survival of islet cell transplants” means to improve the survivability of islet cell transplants by about 5% to 500%, including all whole integers between, e.g., 5%, 6%, 7%, 8%, 9%, 10% . . . 15% . . . 50% . . . 200%, etc. When the survival of islet cell transplants is enhanced, failure or loss of transplanted islet cells is reduced by 1% to 100%, including all whole integers between, e.g., 5%, 6%, 7%, 8%, 9%, 10% . . . 15% . . . 50% . . . 100%, etc.

Each embodiment disclosed herein is contemplated as being applicable to each of the other disclosed embodiments. Thus, all combinations of the various elements described herein are within the scope of the invention.

This invention will be better understood by reference to the Experimental Details which follow, but those skilled in the art will readily appreciate that the specific experiments detailed are only illustrative of the invention as described more fully in the claims which follow thereafter.

### EXPERIMENTAL DETAILS

### Peptide Synthesis and Purification

Human IAPP was synthesized on a 0.1 mmol scale using a CEM Liberty microwave peptide synthesizer utilizing Fmoc chemistry. Solvents used were ACS-grade. The methods have been described previously (Abedini et al. 2005; Marek et al. 2010). In order to afford amidated C terminus peptide, 5-(4′-fmoc-aminomethyl-3′,5-dimethoxyphenol) valeric acid (Fmoc-PAL-PEG-PS) resin was used and purchased from Life Technologies. Standard Fmoc reaction cycles were used. Fmoc protected pseudoproline dipeptide derivatives were incorporated at positions 9-10, 19-20, and 27-28 to facilitate the synthesis. The p-branched residues, Arg, and all pseudoproline dipeptide derivatives were double coupled. A maximum temperature of 50° C. was used for the coupling of His and Cys in order to reduce the possibility of racemization. Peptides were cleaved from the resin by standard trifluoroacetic acid (TFA) methods; ethanedithiol, thioanosole and anisole were used as scavengers. Crude peptides were partially dissolved in 20% acetic acid (v/v), frozen in liquid nitrogen, and lyophilized to increase their solubility. The dry peptide was redissolved in 100% dimethyl sulfoxide (DMSO) at room temperature to promote the formation of the disulfide bond (Abedini at al. 2006; Tam at al. 1991). Peptides were purified by reverse-phase HPLC using a Proto 300 C18 preparative column (10 mm×250 mm). A two buffer gradient was used: buffer A consisted of 100% H2O and 0.045% HCl (v/v) and buffer B included 80% acetonitrile, 20% H2O and 0.045% HCl. HCl was used as the counterion instead of TFA since residual TFA can influence amyloid formation. MALDI-TOF mass spectrometry confirmed the correct molecular weight. (expected 3903.3 Da, observed 3902.8 Da)

### Sample Preparation

Peptides were first dissolved in 100% hexafluoroisopropanol (HFIP) at a concentration 1.6 mM and then filtered to remove any preformed amyloid aggregates. For thioflavin-T fluorescence assays, aliquots were lyophilized and redissolved in pH 7.4, 20 mM Tris-HCl buffer at the desired concentration. Aspirin and Ketoprofen were prepared in 100% dimethyl sulfoxide (DMSO).

### Thioflavin-T Fluorescence Assays

Solutions were prepared by adding pH 7.4, 20 mM Tris buffer and thioflavin-T to lyophilized dry peptides for a final peptide concentration of 16 M. For the studies of Aspirin and Ketoprofen, 0.25% DMSO was present in the solution. Measurements were made at 25° C. using a Beckman Coulter DTX880 plate reader without stirring. An excitation filter of 430 nm and an emission filter of 485 nm were used. To test disaggregate activity of Aspirin, peptide was first incubated in low-binding 96-wells plate and monitored by plate reader to ensure the formation of amyloid fibrils.

### Right Angle Light Scattering Assays (RALS)

Solutions were prepared by adding pH 7.4, 20 mM Tris buffer without thioflavin-T to lyophilized dry peptides for a final peptide concentration of 16 solutions were prepared by adding pH 7.4, 20 mM Tris buffer without thioflavin-T to lyophilized dry passays used an excitation and emission wavelength of 500 nm.

### Transmission Electron Microscopy (TEM)

TEM was performed at the Life Science Microscopy Center at Stony Brook University. Aliquots were removed from the same solutions that were used for the fluorescence measurements. 5 μL of peptide solution was placed on carbon-coated Formvar 300 mesh copper grid for one minute and then negatively stained with saturated uranyl acetate for another one minute.

### Example 1

### Effect of Flurbiprofen on Human IAPP Amyloid Formation

Flurbiprofen accelerated islet amyloid formation and reduced the presence and toxic effects of intermediates of amyloid formation in a dose dependent fashion. The highest concentration of OH-flurbiprofen (20 to 1 ratio of flurbiprofen to human IAPP) showed the highest rate of acceleration of amyloid formation.

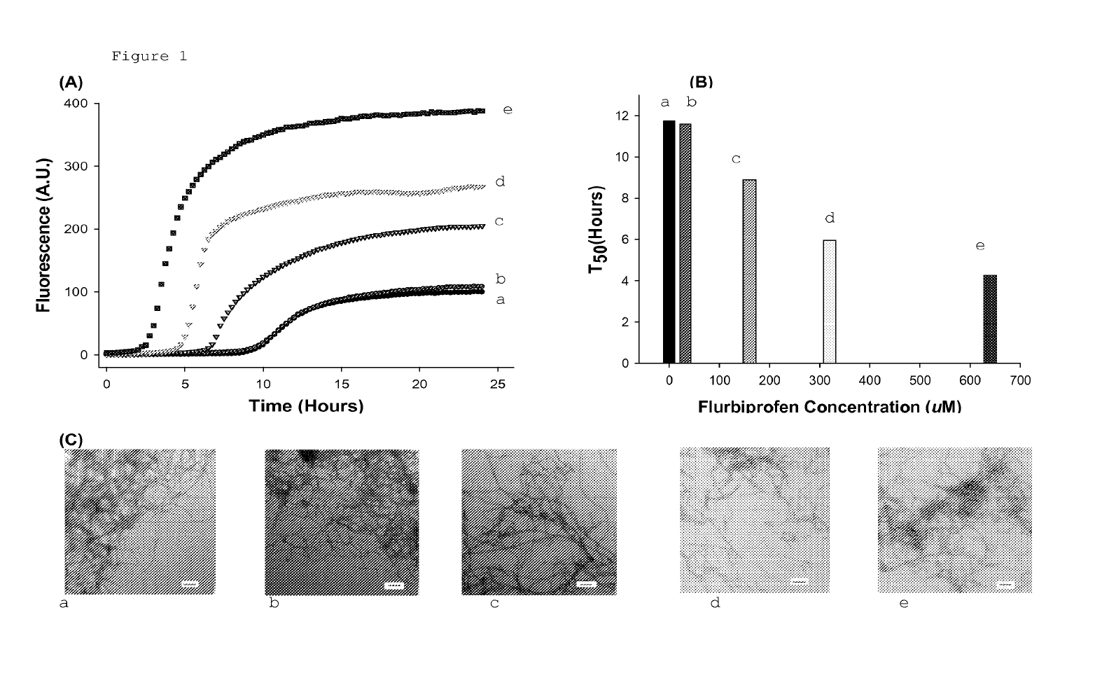

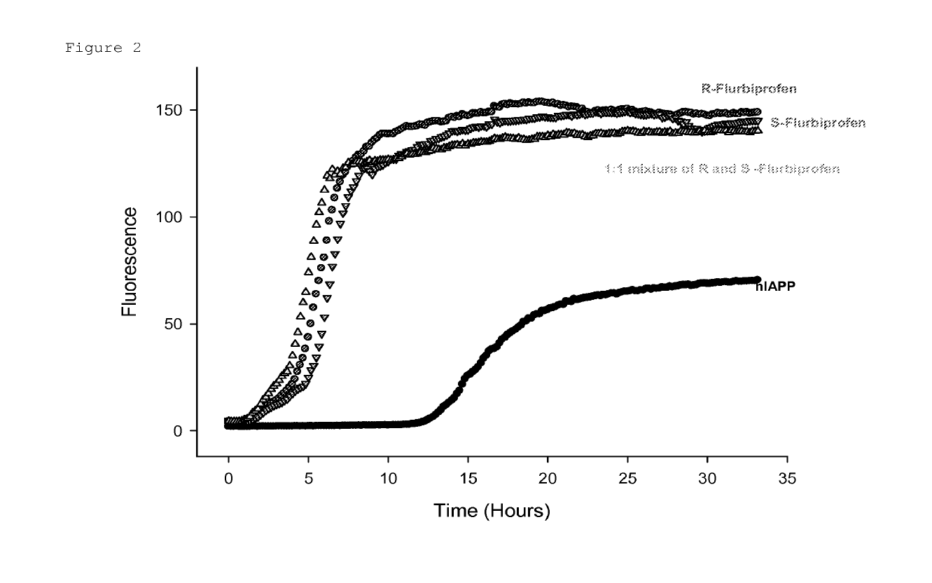

A mixture of flurbiprofen (1-20 parts) and human IAPP (1 part) was monitored by fluorescence detected thioflavin-T assays. The assays monitored the time course of amyloid formation (amount vs time) in the presence and absence of flurbiprofen. Transmission electron microscopy was used to confirm the presence of amyloid (FIG. 1).

Flurbiprofen racemate, R-flurbiprofen and S-flurbiprofen each accelerated islet amyloid formation and reduced the presence and toxic effects of intermediates of amyloid formation. The racemic mixture and the R and S enantiomers were equally effective (FIG. 2).

### Example 2

### Flurbiprofen Induces Human IAPP to Form Amyloid Deposits that Closely Resemble Those Found in the Absence of the Compound

One and two dimensional infrared (IR) spectroscopy was used to confirm the presence of beta-sheet structure. Amyloid is rich in beta-sheet structure. IR spectra were recorded of a mixture of 0.5 mm human IAPP and 10 mM flurbiprofen.

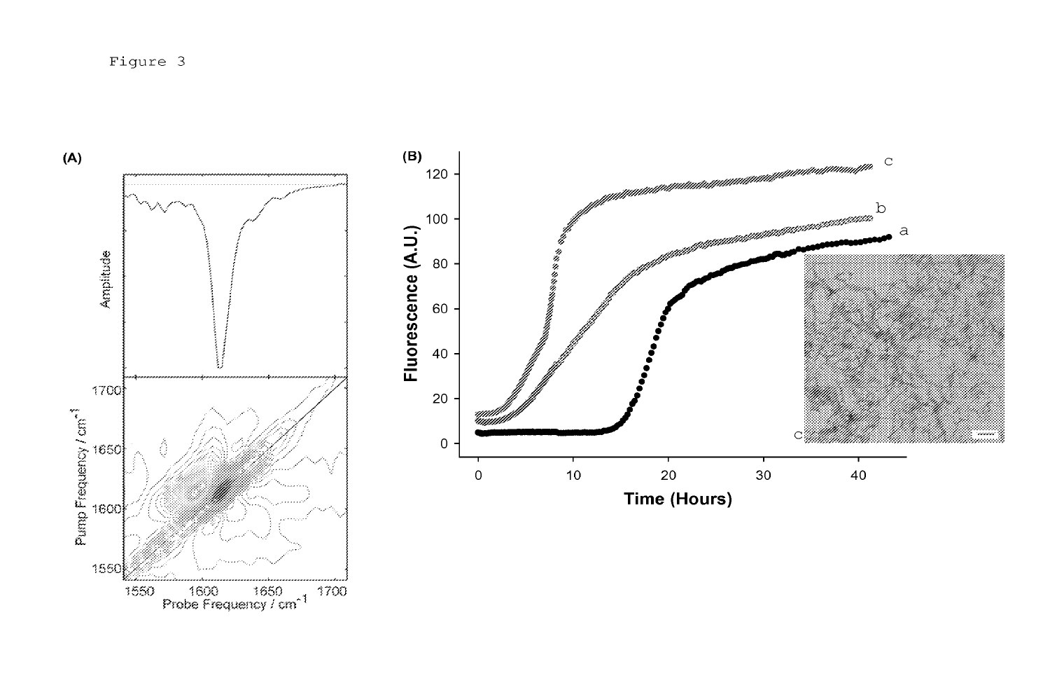

Seeding experiments were conducted using the products of the experiment conducted with the mixture of flurbiprofen (20 parts) and human IAPP. Seeding refers to the addition of a small amyloid of pre-formed amyloid fibers to an initially unaggerated solution of polypeptide. Addition of the amyloid fibers leads to a by passing of the lag phase. Seeding is usually specific in that amyloid formed by one protein usually does not seed amyloid formation by a different protein. 10% seeds, by mass, were added to unaggerated human IAPP and amyloid formation monitored by fluorescence detected thioflavin-T assays. The mixture of flurbiprofen (20 parts) and human IAPP was extremely effective at seeding amyloid formation by human IAPP. The highly efficient seeding indicates that the structure of the amyloid fibers formed in the presence of flurbiprofen is very similar to the structure of the amyloid formed in its absence. Results are shown in FIG. 3.

### Example 3

### Effect of 2-(2-fluoro-4′-hydroxybiphenyl-4-yl)propanoic Acid (OH-flurbiprofen) On Human IAPP Amyloid Formation

A mixture of OH-flurbiprofen and human IAPP were monitored by fluorescence detected thioflavin-T assays. Three different concentrations (1:1 ratio, 1:10 ratio and 1:20 ratio of IAPP to OH-flurbiprofen) were tested against human IAPP without any added OH-flurbiprofen. The assays monitored the time course of amyloid formation (amount vs time) in the presence and absence of a flurbiprofen derivative.

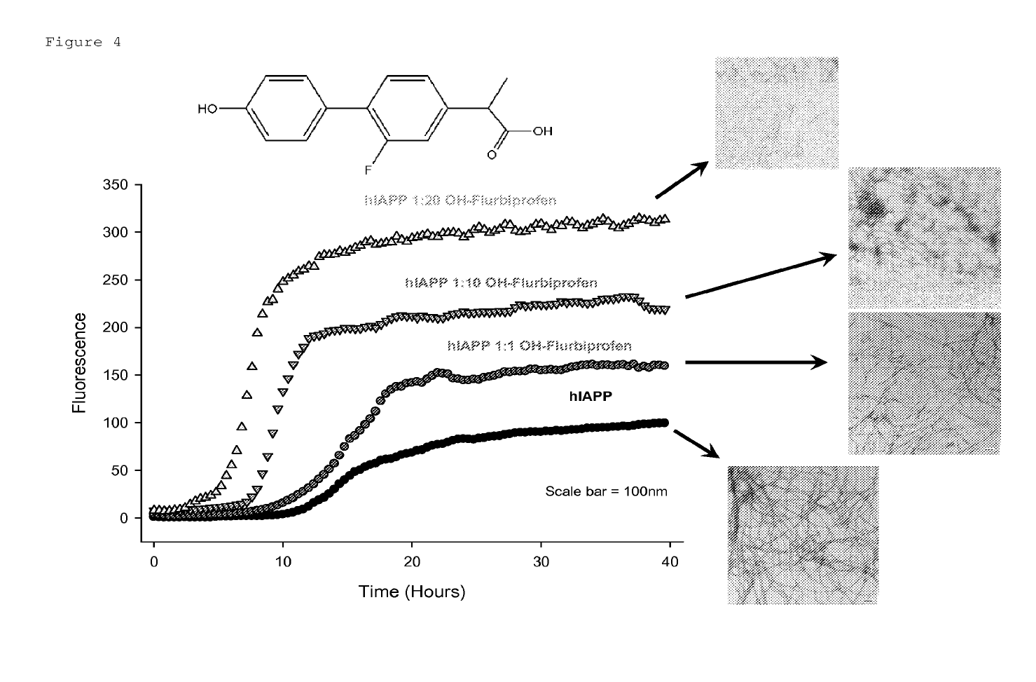

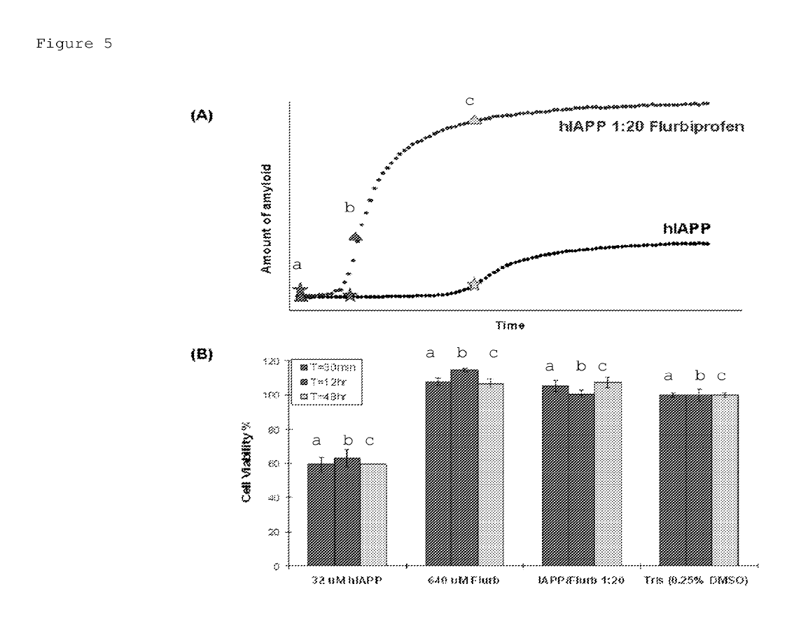

OH-Flurbiprofen accelerated islet amyloid formation and reduced the presence and toxic effects of intermediates of amyloid formation in a dose dependent fashion. The highest concentration of OH-flurbiprofen (20 to 1 ratio of OH-flurbiprofen to human IAPP) showed the highest rate of acceleration of amyloid formation. A 1:1 ratio of human IAPP and flurbiprofen also showed acceleration of amyloid formation. Samples were analyzed by electron microscopy. Results are shown in FIG. 4.

### Example 4

### A Direct Correlation Between the Lifetime of Amyloidogenic Intermediates and Duration of Toxicity

Parallel biophysical and cell viability experiments were conducted using a range of conditions that alter the length of the lag phase. h-IAPP amyloid kinetics are concentration-dependent. Decreasing the peptide concentration leads to an increase in the length of the lag phase, while an increase in human-IAPP concentration leads to a shortening of the lag phase. The length of the lag phase can also be controlled by altering the temperature. Lower temperatures lead to a longer lag phase; the lag phase is 2-fold longer at 15° C. Mutations that slow the onset of amyloid formation, but do not prevent formation of toxic intermediates were tested. A set of rationally designed Ser-20 mutations were used. Ser-20 is located at a critical position in the h-IAPP sequence and modulates its aggregation kinetics. Substitution of Ser-20 with a Gly residue increases the rate of amyloid formation and abolishes the lag phase, while substitution with a Lys significantly increases the length of the lag phase, decreasing the rate of amyloid formation (Cao at al. 2012b). Toxicity and kinetics experiments were performed on the Ser-20 mutants. The faster rate of aggregation of S20G-IAPP led to a more rapid onset and shorter duration of toxicity relative to wild type human IAPP. On the other hand, the slower aggregation rate of S20K-human IAPP increased the duration of toxicity. An human-IAPP triple mutant, 3×L-IAPP, in which Leu replaced the three aromatic residues (Phe-15, Phe=23 and Tyr-37) was tested. This mutant forms amyloid 9-fold more slowly than wild type human IAPP (Marek at al. 2007). The triple mutant led to a longer duration of toxicity than that observed with wild type. The duration of toxicity for all the mutants and for all the conditions with human IAPP wild type correlated with the length of the respective lag phases.

Plotting the length of the lag phase for all of the variants of h-IAPP versus their respective duration of toxicity reveals a striking linear correlation, supporting the direct relationship between the kinetics of amyloid formation and the duration of toxicity.

### Example 5

### Effect Of 2-(biphenyl-4-yl)acetic acid on IAPP amyloid formation

A mixture of 2-(biphenyl-4-yl)acetic acid (20 parts) and human IAPP (1 part) are monitored by fluorescence detected thioflavin-T assays.

The assays monitor the time course of amyloid formation (amount vs time) in the presence and absence of a flurbiprofen derivative.

2-(Biphenyl-4-yl)acetic acid accelerates islet amyloid formation and reduces the presence and toxic effects of intermediates of amyloid formation.

### Example 6

### Effect of (S)-2-(biphenyl-4-yl)propanoic acid on IAPP amyloid formation

A mixture of (S)-2-(biphenyl-4-yl)propanoic acid (20 parts) and human IAPP (1 part) are monitored by fluorescence detected thioflavin-T assays. The assays monitor the time course of amyloid formation (amount vs time) in the presence and absence of a flurbiprofen derivative.

(S)-2-(Biphenyl-4-yl)propanoic acid accelerates islet amyloid formation and reduces the presence and toxic effects of intermediates of amyloid formation.

### Example 7

### Effect of 2-(2-fluorobiphenyl-4-yl)acetic acid on IAPP amyloid formation

A mixture of 2-(2-fluorobiphenyl-4-yl)acetic acid (20 parts) and human IAPP (1 part) are monitored by fluorescence detected thioflavin-T assays. The assays monitor the time course of amyloid formation (amount vs time) in the presence and absence of a flurbiprofen derivative.

2-(2-Fluorobiphenyl-4-yl)acetic acid accelerates islet amyloid formation and reduces the presence and toxic effects of intermediates of amyloid formation.

### Example 8

### Acceleration of Amyloid Formation Using Flurbiprofen or a Flurbiprofen Derivative Results in Decreased Presence of Toxic Intermediates

The presence of toxic intermediates during accelerated and non-accelerated amyloid formation is analyzed. Accelerated amyloid formation is induced by flurbiprofen or a flurbiprofen derivative.

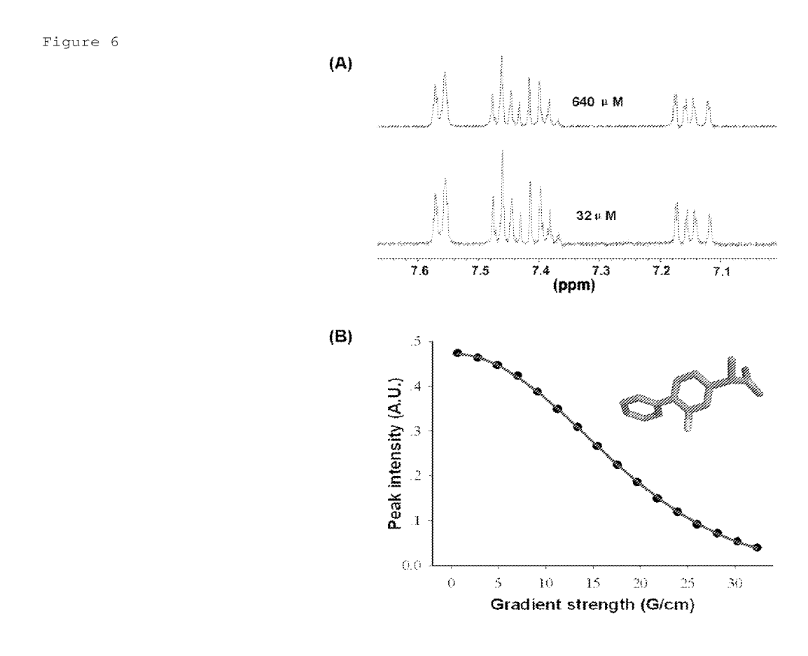

The presence of toxic intermediates is reduced when amyloid formation is accelerated by use of flurbiprofen or a flurbiprofen derivative. A thioflavin-T monitored time course of amyloid formation by human IAPP was conducted in the presence and absence of flurbiprofen. Aliquots were removed at various time points and applied to rat INS-1 beta-cells; at thirty minutes, at 12 hours, at 48 hours. Cell viability as judged by AlamarBlue assays. 20 mM Tris-HCl buffer with 0.25% DMSO was used as the control. Results are shown in FIG. 5.

### Example 9

### Treatment of the Toxic Effects of Amyloid Formation Using Flurbiprofen or a Flurbiprofen Derivative in Human IAPP Transgenic Mouse Islets

Amyloid formation is induced in human IAPP transgenic mouse islets in the presence and absence of flurbiprofen or a flurbiprofen derivative.

The presence of flurbiprofen or a flurbiprofen derivative reduces the toxic effects of amyloid formation.

### Example 10

### Administration of Flurbiprofen and Flurbiprofen Derivatives to a Mouse Receiving Islet Cell Transplants

Mice transplanted with human islet cell transplants are treated with flurbiprofen or a flurbiprofen derivative. Flurbiprofen or a flurbiprofen derivative enhances the survival of the islet cell transplants.

### Example 11

### Effect of IAPP Mutants on the Toxic Effects of Amyloid Formation by Wild Type Human IAPP

The toxic effects of amyloid formation induced by human wild type IAPP are analyzed in the presence and absence of a mutated form of human IAPP having single proline mutations in the 20-29 region.

The presence of a mutated form of human IAPP having single proline mutations in the 20-20 region prolongs the toxic effects of amyloid formation by human wild type IAPP.

### Example 12

### Flurbiprofen does not Aggregate

Flurbiprofen was dissolved at 32 micro molar and at 640 micromolar and Nuclear Magnetic Resonance (NMR) spectra were recorded. The peak positions and line widths are independent of the concentration of the compound.

A sample of 640 micromolar Flurbiprofen was prepared and pulse field gradient NMR experiments were conducted to determine the diffusion coefficient of the compound. The diffusion coefficient is directly related to the hydrodynamic radius of the compound. The NMR intensity decay curve was monitored for the methyl group NMR signal. The best fit to the data yields a hydrodynamic radius of 3.9 Å.

Results are shown in FIG. 6. Flurbiprofen did not aggregate.

### Example 13

### Flurbiprofen does not Induced Amyloid Formation by Non-Amyloidogenic and Non-Toxic Variants of IAPP

The I26P human IAPP point mutant was incubated with Flurbiprofen (20 fold excess of Flurbiprofen) and the mixture was monitored by fluorescence detected thioflavin-T assays. No increase in fluorescence is detected; indicating that amyloid is not formed. Transmission electron microscopy was used to confirm the absence of amyloid.

The I26P human IAPP point mutant was incubated with 100 nm vesicles composed of 100% anionic lipids as a control experiment. The mixture was monitored by fluorescence detected thioflavin-T assays. An increase in fluorescence is detected; indicating that amyloid is formed. Transmission electron microscopy was used to confirm the presence of amyloid. This control experiment shows negatively charged lipid vesicles can induce amyloid formation.

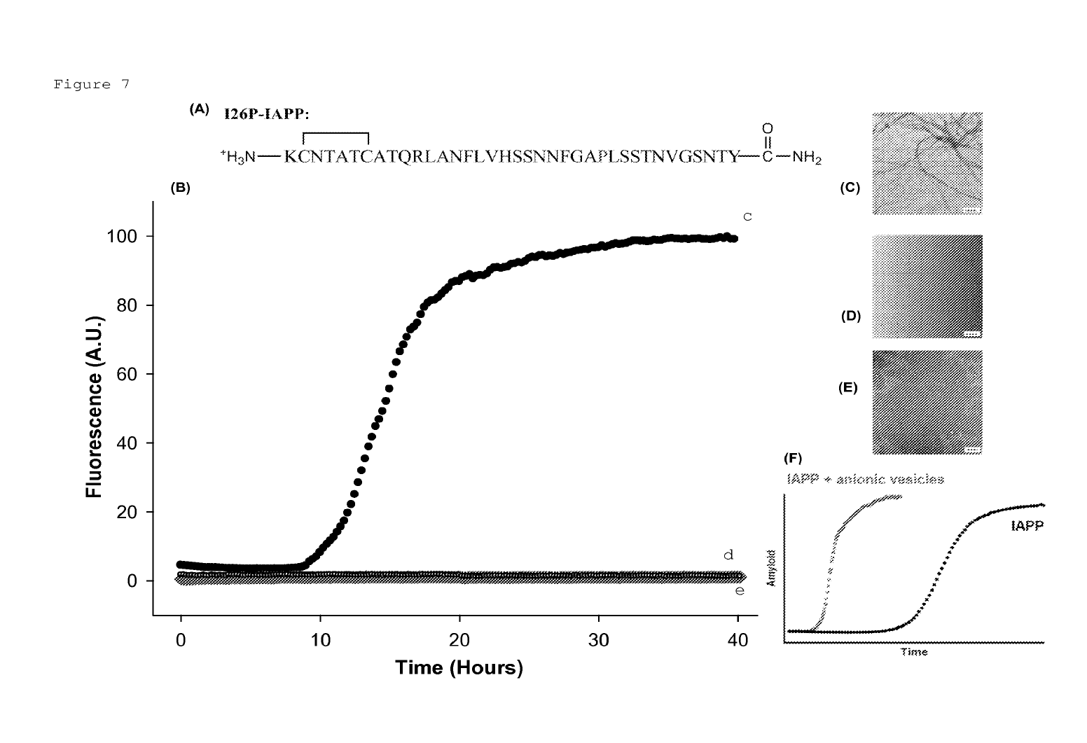

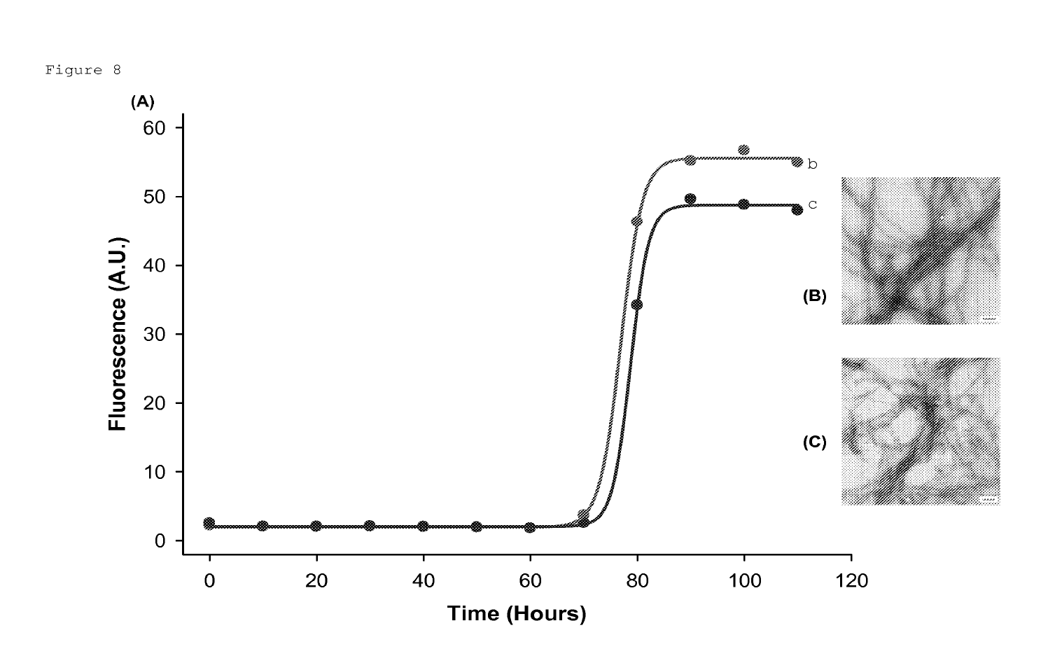

Results are shown in FIG. 7. Flurbiprofen did not induced amyloid formation by non-amyloidogenic and non-toxic variants of IAPP.

### Example 14

### Effect of Flurbiprofen on A-Beta(1-40) Amyloid Formation

A mixture of flurbiprofen (20 parts) and A-beta(1-40) (1 part) was monitored by fluorescence detected thioflavin-T assays. The assays monitored the time course of amyloid formation (amount vs time) in the presence and absence of flurbiprofen.

Flurbiprofen did not influence the kinetics of amyloid formation by A-beta(1-40). Flurbiprofen did not accelerated A-beta(1-40) amyloid formation.

Samples were analyzed by electron microscopy. Results are shown in FIG. 8.

### Example 15

### Effect of Flurbiprofen on Mutant Human IAPP Amyloid Formation

A mixture of flurbiprofen (20 parts) and human IAPP (1 part) was monitored by fluorescence detected thioflavin-T assays. The assays monitored the time course of amyloid formation (amount vs time) in the presence and absence of flurbiprofen. The effect of flurbiprofen in wild-type (WT) human IAPP, and in two mutants of human IAPP was monitored. In one mutant, all of the aromatic amino acids in human IAPP were replaced by lecine: F15L, F23L, Y37L human IAPP was examined. A second mutant in which histidine-18 was replaced with glutamine (H18Q human IAPP) was also tested.

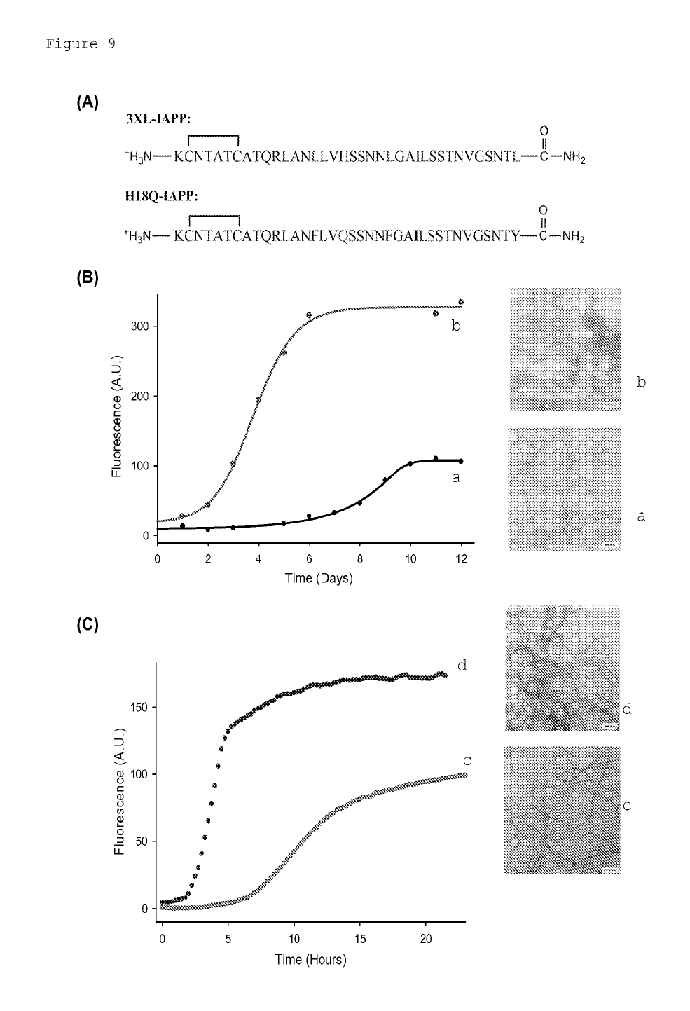

In the first mutant, all three aromatic groups of IAPP were replaced by Leucine (F15L, F23L, Y37L). Flurbiprofen had the same effect on the IAPP mutant having all three aromatic groups replaced by Leucine (F15L, F23L, Y37L) as it did on wild type. This effect is important because some amyloid inhibitors work by binding to aromatic groups in protein. Our data shows this is not true for flurbiprofen. Results are shown in FIG. 9.

In the second mutant, His-18 was replaced by Glutamine (H18Q). The compound effected the mutant the same way as it did the wild type. Results are shown in FIG. 9. Although interactions with His have been suggested to be important for some other inhibitors, this data shows this is not the case for flurbiprofen.

Flurbiprofen racemate, R-flurbiprofen and S-flurbiprofen accelerate islet amyloid formation and reduce the toxic effects of intermediates of amyloid formation about equally for amyloids formed from WT human IAPP and mutant IAPP.

### Example 16

### Effect of Flurbiprofen on Human IAPP Amyloid Formation Compared to Other NSAIDS

Three different mixtures of NSAIDs (20 parts) and human IAPP (1 parts) were compared to each other and human IAPP alone by fluorescence detected thioflavin-T assays. The assays monitored the time course of amyloid formation (amount vs time) in the presence and absence of aspirin, naproxen and flurbiprofen.

Flurbiprofen showed the strongest effect of accelerating human IAPP amyloid formation. No other non-steroidal anti-inflammatory drug (NSAID) accelerated human IAPP amyloid formation as efficiently as flurbiprofen. Samples were analyzed by electron microscopy.

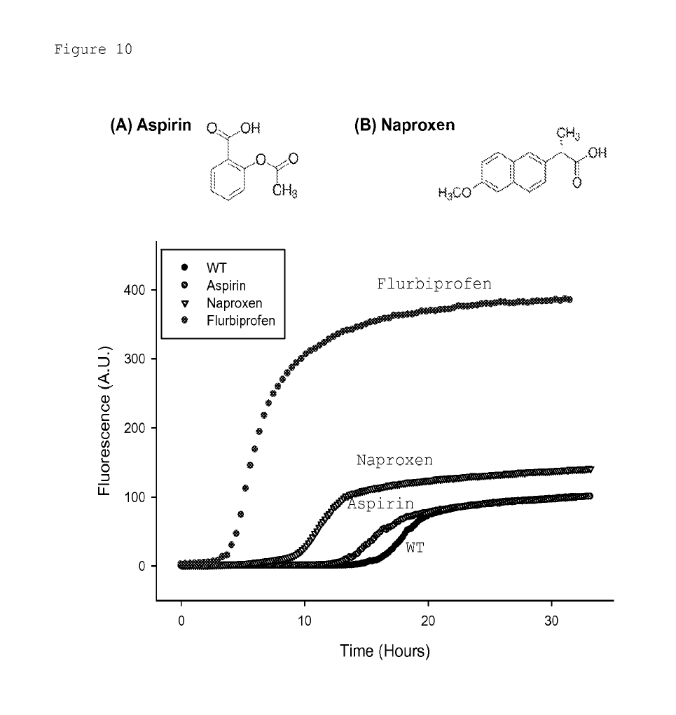

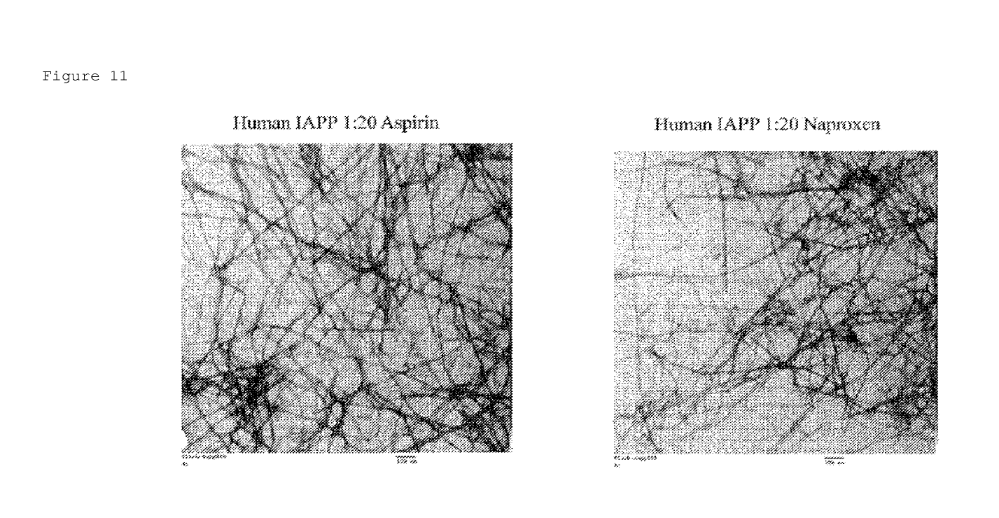

Electron microscopy showed that Aspirin and Naproxen showed no changes in amyloid formation. Results are shown in FIG. 10.

### Example 17

### Aspirin and Ketoprofen

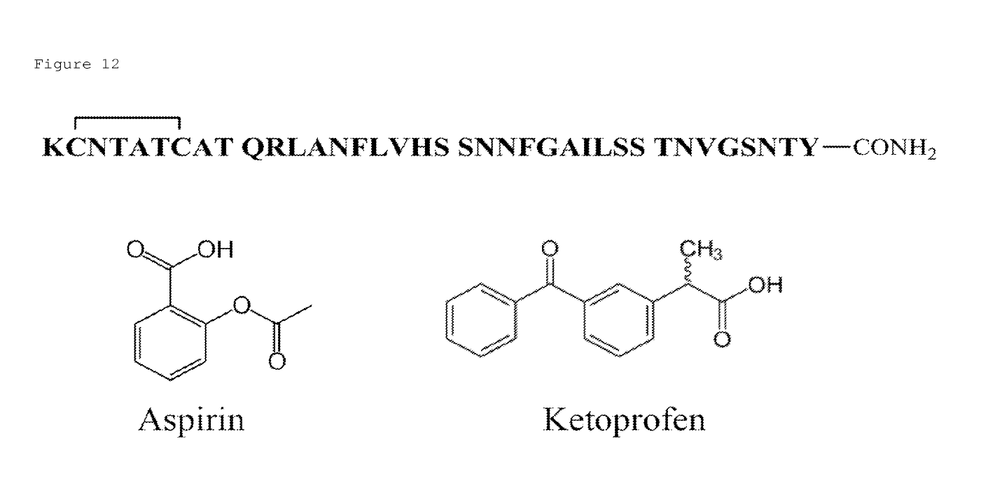

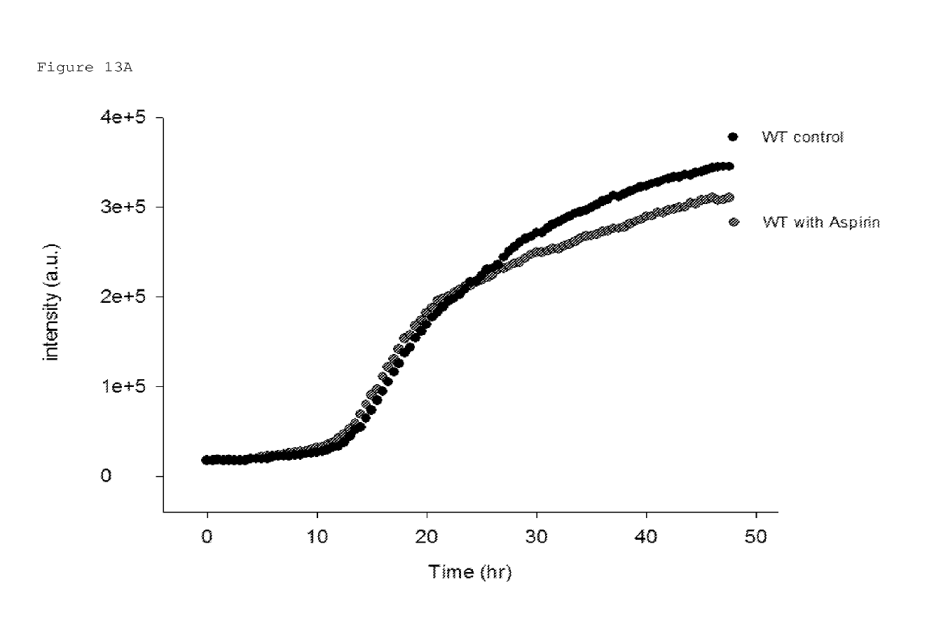

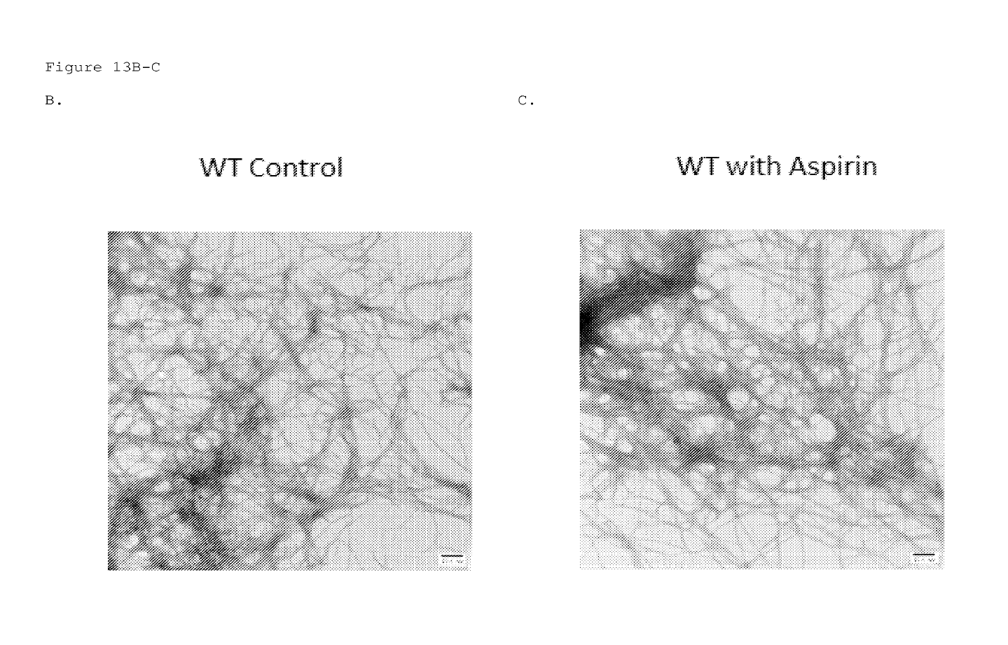

Mature, fully processed, Amylin is 37 residues in length, contains a disulfide bond between residues 2 and 7 and has an amidated C-terminus (FIG. 12). The effects of aspirin on the kinetics of amyloid formation were examined using fluorescence detected thioflavin-T binding assays. Thioflavin-T is a small fluorescent dye that binds to the cross β-structure of amyloid fibrils, presumably in the surface grooves formed by two parallel β-sheets. Binding constrains the conformation of the dye and relieves self-quenching, resulting in an increase in quantum yield (Levine 1995). FIG. 13 displays the results of kinetic experiments conducted in the presence of aspirin. The expected sigmoidal time course is observed in the absence of aspirin with a T₅₀, defined as the time required reaching half of the total signal change in the thioflavin-T assay, of 20 hours. Addition of aspirin, up to a 20-fold excess, had no detectable effect on the rate of amyloid formation, as judged by the values of T₅₀. The compound also had no detectable effect on the final thioflavin-T intensity.

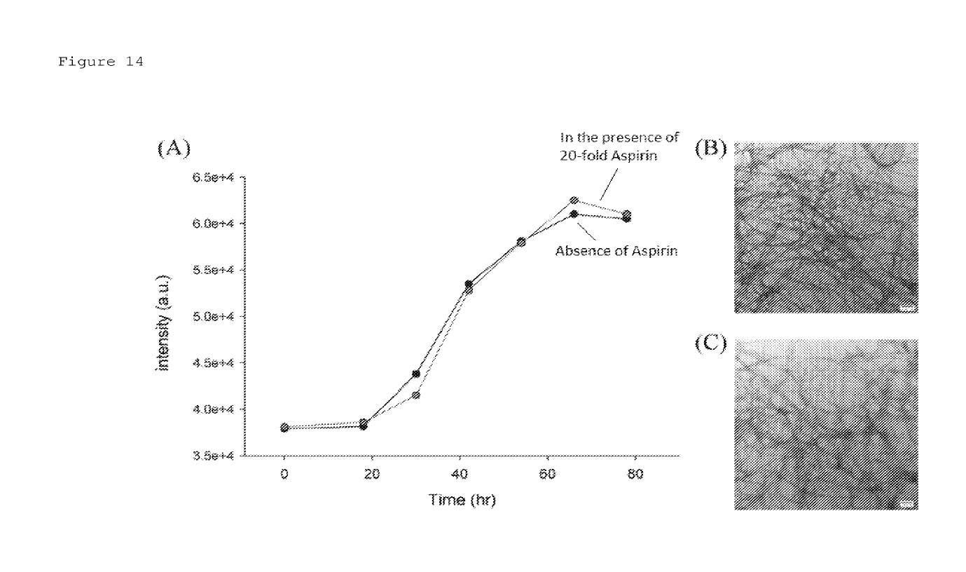

Thioflavin-T binding assays are indirect since they rely on the binding of an extrinsic probe, and can sometimes give misleading results (Meng et al. 2008), but do have the advantage that they report on the kinetics of IAPP amyloid formation in the absence of conflicting factors. Thus, transmission electron microscopy (TEM) was also used to monitor the effects of aspirin. Aliquots were removed from each sample at the end of kinetic experiments, blotted onto TEM grids and imaged. Extensive matts of fibrils were observed in the sample of amylin alone and in the samples containing aspirin. Initial reports of the ability of aspirin to inhibit amylin amyloid formation did not use thioflavin-T assays or TEM, but rather used circular dichroism (CD) and Congo Red binding assays. It is possible, although unlikely, that thioflavin-T could displace aspirin from amylin and interfere with its effects. This does not seem plausible since thioflavin-T has no such effect on a wide range of other inhibitors (Marek at al. 2008; Cao et al. 2012b) and does not bind to pre-amyloid intermediates. None the less, the effects of aspirin in the absence of thioflavin-T were tested using right angle light scattering (RALS) and TEM. RALS reports an observation of similar aggregation curves in the presence and in the absence of a 20-fold excess of aspirin (FIG. 14). TEM reveals the presence of dense matts of fibrils in each sample. Note that these samples did not contain thioflavin-T.

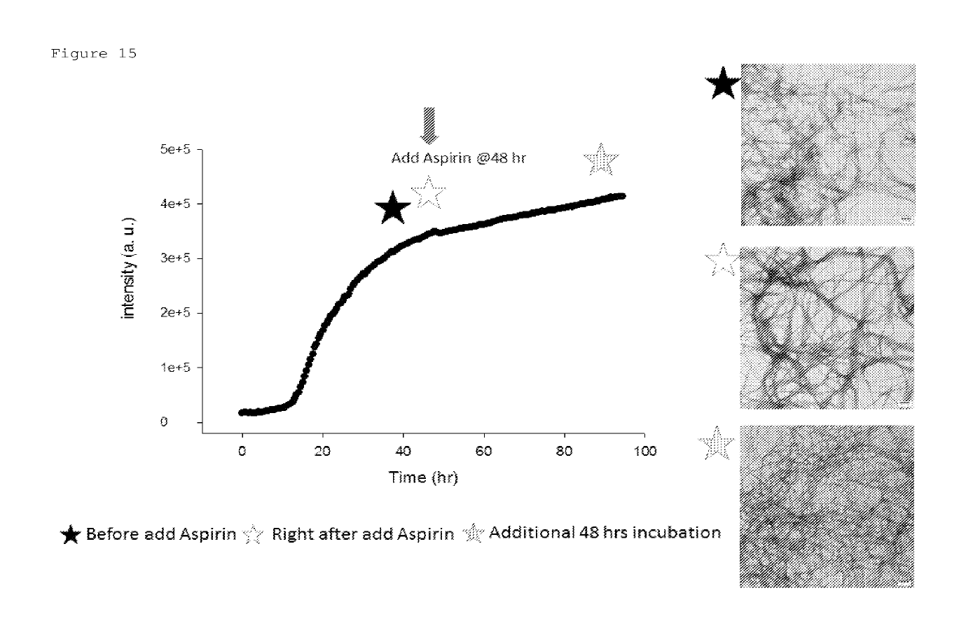

The ability of aspirin to disaggregate pre-formed amylin amyloid fibrils was examined. Some, but not all inhibitors of amyloid formation have this property. Amyloid formation using thioflavin-T assays was monitored (FIG. 15) and 20 fold excess of aspirin was added after amyloid formation was complete and the reaction reached the saturation phase. Aliquots were removed for TEM analysis just before addition of aspirin, immediately afterwards and 48 hours later. Addition of the compound did not perturb the thioflavin T time course, in contrast, compounds which disaggregate amyloid fibrils leads to a decay in the thioflavin-T signal as a function of time (Cao et al. 2012a). The TEM images recorded before and after addition of aspirin are very similar and reveal extensive deposits of amyloid fibrils, confirming that compound does not disaggregate amylin amyloid (FIG. 15B-D).

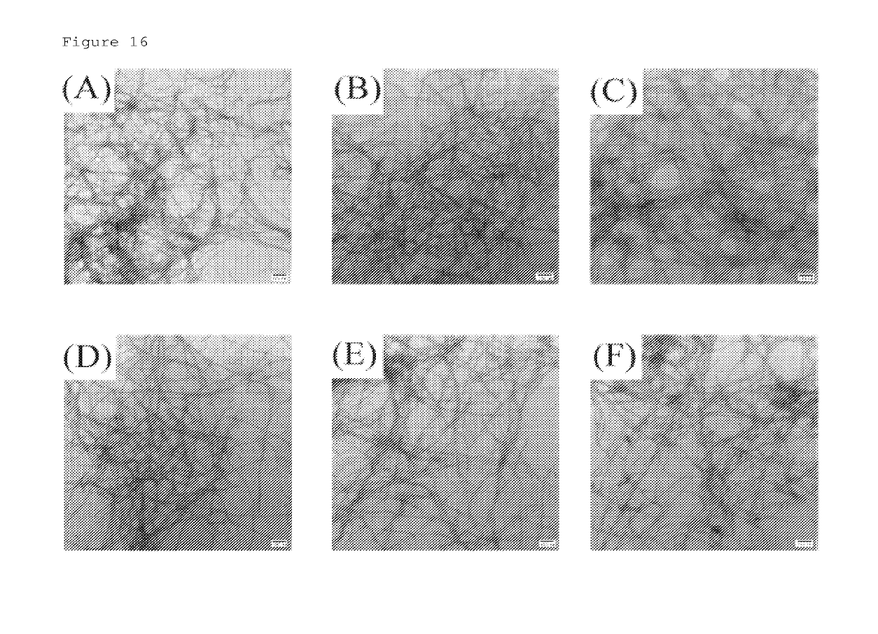

The NSAID Ketoprofen has also been proposed to be an inhibitor of amylin amyloid formation, again on the basis of Cong Red assays and CD spectroscopy. The ability of the compound to inhibit amyloid formation was analyzed by human amylin using TEM to directly test its effect on amyloid formation (FIG. 16). TEM analysis revealed the presence of extensive matte of amyloid fibrils in all samples, confirming that the compound is not an amylin amyloid inhibitor.

Not all non-steroid anti-inflammatory drugs affect amyloid formation by IAPP. The data presented in FIGS. 12-16 demonstrates that the conclusions by Thomas et al. 2003 were incorrect. In that paper the authors incorrectly concluded that aspirin inhibited amyloid formation by IAPP. The authors used CD and claimed that it showed that aspirin perturbed beta sheet formation (amyloid is rich in beta sheet), but the published CD spectra actually are completely consistent with beta sheet formation. There is a slight reduction of intensity in the CD spectrum in the presence of aspirin, but that is caused by interference from the aspirin not because of changes in the structure of IAPP. The authors also used Congo Red staining, an indirect method, to purportedly shown changes in the amount of amyloid. However Congo Red is not quantitative and the presence of aspirin may interfere with the assay. In contrast, TEM is not subjected to interfering effects from aspirin and is a direct measure of the presence or absence of amyloid.

The TEM image presented in FIG. 14 conclusively shows that aspirin does not inhibit IAPP amyloid formation. Thomas et al. also claimed, incorrectly that aspirin could reverse amyloid formation. The data presented herein (FIG. 15) shows that this is incorrect. FIG. 15 shows the effect of adding aspirin to pre-formed IAPP amyloid fibers. Addition of the compound has no effect on the thioflavine-T signal and TEM shows that large amounts of amyloid are still present even after addition of a 20-fold excess of aspirin.

### DISCUSSION

Analogs of human amylin (h-amylin) which are less aggregation prone than wild-type h-amylin have been approved as an adjunct to insulin therapy (Hollander et al. 2003), but there is no treatment for islet amyloidosis, nor are there any approved therapeutic strategies to prevent islet amyloid deposition. The search for inhibitors of amyloid aggregation and amyloid formation is an active area of research (Cao et al. 2012a; Meng et al. 2010a, 2011; Porat et al. 2004; Mishra et al. 2009; Kapurniotu et al. 2002; Porat et al. 2006; Noor at al. 2012), but relatively anti IAPP amyloid compounds have been developed and the vast majority of those are not drug like. Recently the intriguing possibility that clinically relevant doses of Aspirin and non-steroid anti-inflammatory drug Ketoprofen may inhibit amylin amyloid formation and might disaggregate pre-formed amylin amyloid fibrils has been raised (Thomas et al. 2003). This could potentially open very attractive, inexpensive therapeutic approaches if the compounds were indeed effective anti-amylin amyloid agents. Particularly as inflammation is believed to play a role in islet amyloidosis toxicity (Ehses et al. 2007; Donath et al. 2011; Meier et al. 2014; Masters at al. 2010).

Aspirin and Ketoprofen do not inhibit amylin amyloid formation, even when added at 20-fold excess. The compounds are also unable to disassemble pre-formed amylin amyloid. The differences are due to interference with the analytical assays used. The data presented here show that Aspirin and Ketoprofen do not inhibit amylin amyloid formation under the condition used, pH 7.4 and 25° C. The earlier studies used trifluroethanol (TFE) to promote amyloid formation. TFE and hexafluoroisopropanol (HFIP) stabilize secondary structure on peptides and even modest amount of HFIP or TFE can accelerate amyloid formation (Yanagi et al. 2011). The use of a non-aqueous solvent to induce amyloid formation may contribute to the different conclusions but the methods used also play a role. The previous study made use of CD and absorbance detected Congo red binding assays. The reported CD spectra are clearly different in the presence of high concentration of aspirin, however, the reported spectrum, even at the highest concentration of aspirin, is not that of a random coil, and has a shape consistent with significant β-sheet intensity. In addition, the strong absorbance of aspirin and Ketoprofen in the range of 200-250 nm will interfere with CD measurement. Congo Red binding was also used to test for the presence of amyloid in the original studies. Congo Red staining is a classic method to probe amyloid formation, particularly for ex-vivo amyloid deposits and usually involves monitoring birefringence, but the absorbance-based assays are also used. In either case the dye is an extrinsic probe and it has been shown to not be amyloid specific (Khurana et al. 2001). In the case of absorbance assays, addition of compounds can interfere with the assays by contributing background absorbance or by interfering with the binding of the dye. These considerations and data presented here highlight the importance of using multiple probes to study amyloid inhibition, particularly methods such as TEM which directly detect amyloid fibrils.

Flurbiprofen, however, inhibits the toxic effects of amyloid formation. Amyloid formation follows a complicated time course and multiple toxic species may be produced. Without being limited to any particular mode of action, flurbiprofen inhibits toxic species by modulating the kinetics of amyloid formation.

The kinetics of amyloid formation are complex: the formation of amyloid displays a sigmoidal profile with three observable phases (Chiti et al. 2006). The initial steps of aggregation, which leads to formation of an active seed, occur in the lag phase and are the rate limiting process. In this phase, monomers oligomerize and convert into species that nucleate production of amyloid fibers, but little, or no, amyloid is formed. The lag phase is followed by a “growth phase” during which fibers form and elongate. Fibrils elongate by addition of peptide to their ends. Secondary nucleation that involves the catalyst of fibril formation from existing fibrils also occurs. Eventually, a steady state is reached where soluble peptide is at equilibrium with amyloid fibrils. Off-pathway steps leading to amorphous aggregates can also occur.

It was unexpectedly found that the pre-amyloid intermediates are the toxic species for IAPP amyloid and acceleration of amyloid formation reduces the presence of these toxic intermediates. The Examples above show that flurbiprofen and flurbiprofen derivatives effectively inhibit the toxic effects of the pre-amyloid species by accelerating amyloid formation.

Without being limited to any particular mode of action, the Examples above show that flurbiprofen works by a novel biophysical mechanism in vitro. Flurbiprofen accelerates aggregation, but at the same time surprisingly protects against the toxic effects of the intermediates. Other compounds typically slow the rate of aggregation which can lead to the buildup of toxic intermediates.

Flurbiprofen is the first small drug-like molecule that can be used to inhibit the toxic effects of islet amyloidosis. Islet amyloidosis plays an important role in type 2 diabetes and in the failure of islet cell transplants. Flurbiprofen can be used for the treatment of certain aspects of type 2 diabetes and to prolong the survival of islet cell transplants.

As shown in Example 1, the R and S enantiomers of flurbiprofen and the flurbiprofen racemate are equally effective.

As shown in Example 3, derivatives of flurbiprofen are also effective.

As shown in Example 15, flurbiprofen is effective to inhibit the toxic effects of amyloid formation by mutants of IAPP. There is one mutant of IAPP that is found in humans which occurs at low levels in certain Asian populations and has been suggested to be linked to a modest increased risk of diabetes. Flurbiprofen impacts amyloid formation by a broad range of point mutants and multiple mutants of IAPP.

As shown in Example 16, other NSAIDs do not impact amyloid formation. Amyloid has no connection to the mode of action of NSAIDs. However, a different anti-inflammatory with a very different structure from the compounds of the present invention, salsalate, has been proposed for the treatment of diabetes, but it targets inflammation not amyloid formation and has not been proposed for use in enhancing the survival of islet cell transplants, nor has it been proposed to effect islet amyloid formation (Desouza at al. 2010). Salsalate and its proposed mode of action are fundamentally different from the mode of action demonstrated by the Examples of the present application.

Flurbiprofen has advantages over other IAPP inhibitors because flurbiprofen is a small molecule and is drug-like. Other IAPP inhibitors have disadvantages that preclude such compounds from being attractive as potential drugs. For example, other IAPP inhibitors are either peptides or complex, chemically labile polyphenols, or highly sulfonated compounds with toxicity issues. One reported polypeptide inhibitor, a mutated form of human IAPP having single proline mutations in the 20-29 region, unexpectedly prolongs the toxic effects of IAPP.

Flurbiprofen has advantages over some other compounds because it does not aggregate. Some small molecule inhibitors of amyloid formation work because they aggregate to form micelle/vesicle like structures which hind the amyloidogenic protein. This mode of action is effective in vitro but is not likely to be in vivo (Feng et al. 2008).

### REFERENCES

Abedini, A. & Raleigh, D. P. (2005). Incorporation of pseudoproline derivatives allows the facile synthesis of human IAPP, a highly amyloidogenic and aggregation-prone polypeptide. Org. Lett. 7, 693-696.
Abedini, A. Singh, G. & Raleigh, D. P. (2006). Recovery and purification of highly aggregation-prone disulfide-containing peptides: Application to islet amyloid polypeptide. Anal. Biochem. 351, 181-186.
Abedini, A., Meng, F. L. and Raleigh, D. P. (2007) A single-point mutation converts the highly amyloidogenic human islet amyloid polypeptide into a potent fibrillization inhibitor. J. Am. Chem. Soc. 129, 11300-11301.
Ashcroft, F. M. and Rosman, P. (2012) Diabetes mellitus and the cell: The last ten years. Cell. 148, 1160-1171.
Cao, P. and Raleigh, D. P. (2012a) Analysis of the inhibition and remodeling of islet amyloid polypeptide amyloid fibers by flavanols. Biochemistry 51, 2670-2683.
Cao, P., Tu, L. H., Abedini, A., Levsh, O., Akter, R., Patsalo, V., Schmidt, A. M. and Raleigh, D. P. (2012b) Sensitivity of amyloid formation by human islet amyloid polypeptide to mutations at residue 20. J. Mol. Biol. 421, 282-95.
Cheng, B. A., Gong, H., Li, X. C., Sun, Y., Mang, X., Chen, H., Liu, X. R., Zheng, L. and Huang, K. (2012) Silibinin inhibits the toxic aggregation of human islet amyloid polypeptide. Biochem. Biophys. Res. Commun. 419, 495-499.
Chiti, F., and Dobson, C. M. Protein misfolding, functional amyloid, and human disease, Annu Rev Biochem (2006) 75, 333-366.
Clark, A., Wells, C. A., Buley, I. D., Cruickshank, J. K., Vanhegan, R. I., Matthews, D. R., Cooper, G. J. S., Holman, R. R. and Turner, R. C. (1988) Islet amyloid, increased alpha-cells, reduced beta-cells and exocrine fibrosis—quantitative changes in the pancreas in type-2 diabetes. Diabetes Res. Clin. Ex. 9, 151-159.
Cooper, G. J. S., Willis, A. C., Clark, A., Turner, R. C., Sim, R. B. and Reid, K. B. M. (1987) Purification and characterization of a peptide from amyloid-rich pancreases of type-2 diabetic-patients. Proc. Natl. Acad. Sci. U.S.A 84, 8628-8632.
Desouza, C. V. (2010) An overview of salsalate as a potential antidiabetic therapy drugs of today. 46, 847-853 (2010)
Donath, M. Y. & Shoelson, S. E. (2011). Type 2 diabetes as an inflammatory disease. Nat. Rev. Immunol. 11, 98-107.
Ehses, J. A., Perren, A., Eppler, E., Ribaux, P., Pospisilik, J. A., Maor-Cahn, R., Gueripel, X., Ellingsgaard, H., Schneider, M. K., Biollaz, G., Fontana, A., Reinecke, M., Homo-Delarche, F., & Donath, M. Y. (2007) Increased number of islet-associated macrophages in type 2 diabetes. Diabetes 56, 2356-2370,
Feng B Y, Toyama B H, Wille H, Colby D W, Collins S R, May B C, Prusiner S B, Weissman J, Shoichet B K. (2008) Small-molecule aggregates inhibit amyloid polymerization. Nat Chem Biol 4 (3), 197-9.
Hebda, J. A., Saraogi, I., Magzoub, M., Hamilton, A. D. and Miranker, A. D. (2009) A peptidomimetic approach to targeting pre-amyloidogenic states in type II diabetes. Chem. Biol. 16, 943-950.
Hollander, P. A., Levy, P., Fineman, M., Maggs, D. G., Shen, L. Z., Strobel, S. A., Meyer, C. & Kolterman, 0. G. (2003). Pramlintide as an adjunct to insulin therapy improves long-term glycemic and weight control in patients with type 2 diabetes—A 1-year randomized controlled trial. Diabetes Care 26, 784-790.
Hull, R. L., Westermark, G. T., Westermark, P. and Kahn, S. E. (2004) Islet amyloid: A critical entity in the pathogenesis of type 2 diabetes. J. Clin. Endocrinol. Metab. 89, 3629-3643.
Kahn, S. E., Dalessio, D. A., Schwartz, M. H. Fujimoto, W. Y., Ensinck, J. W., Taborsky, G. J. & Porte, D. (1990). Evidence of cosecretion of islet amyloid polypeptide and insulin by beta-cells. Diabetes 39, 634-638.
Kapurniotu, A., Schmauder, A. & Tenidis, K. (2002). Structure-based design and study of non-amyloidogenic, double N-methylated IAPP amyloid core sequences as inhibitors of IAPP amyloid formation and cytotoxicity. J. Mol. Biol. 315, 339-350.
Khurana, R., Uversky, V. N., Nielsen, L. & Fink, A. L. (2001). Is Congo red an amyloid-specific dye? J. Biol. Chem. 276, 22715-22721.
Konarkowska, B., Aitken, J. F., Kistler, J., Zhang, S. P. & Cooper, G. J. S. (2006). The aggregation potential of human amylin determines its cytotoxicity towards islet beta-cells. FEBS. J. 273, 3614-3624.
Levine, H. (1995). Thioflavine-T interaction with amyloid beta-sheet structures. Amyloid 2, 1-6.
Lorenzo, A., Razzaboni, B., Weir, G. C. and Yankner, B. A. (1994) Pancreatic-islet cell toxicity of amylin associated with type-2 diabetes-mellitus. Nature 368, 756-760.
Marek, P., Abedini, A., Song, B. B., Kanungo, M., Johnson, M. E., Gupta, R., Zaman, W., Wong, S. S. and Raleigh, D. P. (2007) Aromatic interactions are not required for amyloid fibril formation by islet amyloid polypeptide but do influence the rate of fibril formation and fibril morphology. Biochemistry 46, 3255-3261.
Marek, P., Gupta, R. & Raleigh, D. P. (2008). The fluorescent amino acid p-cyanophenylalanine provides an intrinsic probe of amyloid formation. Chembiochem 9, 1372-1374.
Marek, P., Woys, A. M., Sutton, K., Zanni, M. T. & Raleigh, D. P. (2010). Efficient microwave-assisted synthesis of human islet amyloid polypeptide designed to facilitate the specific incorporation of labeled amino acids. Org. Lett, 12, 4848-4851.
Masters S. L. et al. (2010) Activation of the NLRP3 inflammasome by islet amyloid polypeptide provides a mechanism for enhanced IL-1β in type 2 diabetes. Nat. Immunol. 11, 897-904.
Meier, D. T., Marcos, M., Samarasekera, T., Zraika, S. Hull, R. L. & Kahn G. E., (2014) islet amyloid formation is essential for induction of islet inflammation in high fat fed human islet amyloid polypeptide transgenic mice. Diabetologia In Press.
Meng, F. L., Marek, P., Potter, K. J., Verchere, C. B. & Raleigh, D. P. (2008). Rifampicin does not prevent amyloid fibril formation by human islet amyloid polypeptide but does inhibit fibril thioflavin-T interactions: Implications for mechanistic studies beta-cell death. Biochemistry 47, 6016-6024.
Meng, F. L., Raleigh, D. P. and Abedini, A. (2010a) Combination of kinetically selected inhibitors in trans leads to highly effective inhibition of amyloid formation. J. Am. Chem. Soc. 132, 14340-14342.
Meng, F., Abedini, A., Plesner, A., Verchere, C. B. and Raleigh, D. P. (2010b) The flavanol (−)-epigallocatechin 3-gallate inhibits amyloid formation by islet amyloid polypeptide, disaggregates amyloid fibrils, and protects cultured cells against IAPP-induced toxicity. Biochemistry 49, 8127-33.
Meng, F. Abedini, A. Plesner, A., Middleton, C. T., Potter, K. J., Zanni, M. T., Verchere, C. B. and Raleigh, D. P. (2010c) The sulfated triphenyl methane derivative acid fuchsin is a potent inhibitor of amyloid formation by human islet amyloid polypeptide and protects against the toxic effects of amyloid formation. J. Mol. Biol. 400, 555-66.
Meng, F. L. & Raleigh, D. P. (2011). Inhibition of glycosaminoglycan-mediated amyloid formation by islet amyloid polypeptide and proIAPP processing intermediates. J. Mol. Biol. 406, 491-502.
Mishra, R., Sellin, D., Radovan, D., Gohlke, A. and Winter, R. (2009) Inhibiting islet amyloid polypeptide fibril formation by the red wine compound resveratrol. ChemBioChem 10, 445-449.
Montane, J., Klimek-Abercrombie, A., Potter, K. J., Westwell-Roper, C. and Verchere, C. B. (2012) Metabolic stress, IAPP and islet amyloid. Diabetes Obes. Metab. 14, 68-77.
Roar, H., Cao, P. and Raleigh, D. P. (2012) Morin hydrate inhibits amyloid formation by islet amyloid polypeptide and disaggregates amyloid fibers. Protein Sci, 21, 373-382.
Porat, Y., Mazor, Y., Efrat, S. and Gazit, E. (2004) Inhibition of islet amyloid polypeptide fibril formation: A potential role for heteroaromatic interactions. Biochemistry 43, 14454-14462.
Potter, K. J. at al. (2010) Islet amyloid deposition limits the viability of human islet grafts but not porcine islet grafts. Proc. Natl. Acad. Sci. U.S.A 107, 4305-4310.
Rigacci, S., Guidotti, V., Bucciantini, M., Parri, M., Nediani, C., Cerbai, E., Stefani, M. and Berti, A. (2010) Oleuropein aglycon prevents cytotoxic amyloid aggregation of human amylin. J. Nutr. Biochem. 21, 726-735.
Selkoe, D. J. Cell biology of protein misfolding: The examples of Alzheimer's and Parkinson's diseases (2004) Nature Cell Biol 6, 1054-1061.
Sinha, S. et al. (2011) Lysine-specific molecular tweezers are broad-spectrum inhibitors of assembly and toxicity of amyloid proteins. J. Am. Chem. Soc. 133, 16958-69.
Tam, J. P., Wu, C. R., Liu, W. & Zhang, J. W. (1991). Disulfide bond formation in peptides by dimethyl-sulfoxide—Scope and applications. J. Am. Chem. Soc. 113, 6657-6662.
Thomas, T, Nadackal, G T, Thomas, K. (2003) Aspirin and diabetes: Inhibition of amylin aggregation by nonsteroidal anti-inflammatory drugs. Experimental And Clinical Endocrinology & Diabetes. 111, 8-11.
Westermark, P., Wernstedt, C., Wilander, E., Hayden, D. W., Obrien, T. D. and Johnson, K. H. (1987) Amyloid fibrils in human insulinoma and islets of langerhans of the diabetic cat are derived from a neuropeptide-like protein also present in normal islet cells. Proc. Natl. Acad. Sci. U.S.A 84, 3881-3885.
Westermark, G. T., Westermark, P., Berne, C., Korsgren, O. and Transpla, N.N.C.I. (2008) Widespread amyloid deposition in transplanted human pancreatic islets. N. Engl. J. Med. 359, 977-979.
Westermark, P., Andersson, A. and Westermark, G. T. (2011) Islet amyloid polypeptide, islet amyloid, and diabetes mellitus. Physiol. Rev. 91, 795-826.
Yan, L. M., Tatarek-Nossol, M., Velkova, A., Kazantzis, A. and Kapurniotu, A. (2006) Design of a mimic of nonamyloidogenic and bioactive human islet amyloid polypeptide (IAPP) as nanomolar affinity inhibitor of IAPP cytotoxic fibrillogenesis. Proc. Natl. Acad. Sci. U.S.A 103, 2046-2051.
Yanagi, K. Ashizaki, M. Yagi, H. Sakurai, K., Lee, Y. H. & Goto, Y. (2011). Hexafluoroisopropanol induces amyloid fibrils of islet amyloid polypeptide by enhancing both hydrophobic and electrostatic interactions. J. Biol. Chem. 286, 23959-23966.
Zelus, C., Fox, A. Calciano, A., Faridian, B. S., Nogaj, L. A. and Moffet, D. A. (2012) Myricetin inhibits islet amyloid polypeptide (IAPP) aggregation and rescues living mammalian cells from IAPP toxicity. Open Biochem J 6, 66-70.

1. A method of reducing the presence of toxic intermediates of amyloidosis from islet amyloid polypeptide (IAPP) in a patient, comprising administering a compound to the patient a compound having the structure: 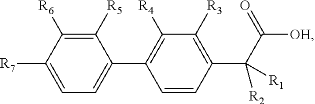
wherein R1 and R2 are each independently hydrogen, methyl, ethyl, propyl, butyl, pentyl or hexyl, or R1 and R2 are linked so as to form a cyclopropyl, cyclobutyl, cyclopentyl, or cyclohexyl ring;
wherein R3, R4, R5, R6 are each independently hydrogen, fluorine or chlorine; and
wherein R7 is hydrogen, phenyl, fluorine, chlorine, bromine, hydroxyl, amine, carboxylic acid, C1-C6 alkyl carboxylate, amide, methyl, ethyl, propyl, butyl, pentyl or hexyl, or
a pharmaceutically acceptable salt or ester thereof,
so as to thereby reduce the presence of toxic intermediates of amyloidosis from islet amyloid polypeptide (IAPP) in the patient.

2. A method of reducing toxicity of amyloidosis from islet amyloid polypeptide (IAPP) in a patient, comprising administering to the patient a compound having the structure: 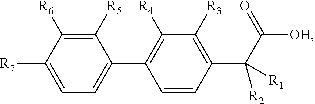
wherein R1 and R2 are each independently hydrogen, methyl, ethyl, propyl, butyl, pentyl or hexyl, or R1 and R2 are linked so as to form a cyclopropyl, cyclobutyl, cyclopentyl, or cyclohexyl ring;
wherein R3, R4, R5, R6 are each independently hydrogen, fluorine or chlorine; and
wherein R7 is hydrogen, phenyl, fluorine, chlorine, bromine, hydroxyl, amine, carboxylic acid, C1-C6 alkyl carboxylate, amide, methyl, ethyl, propyl, butyl, pentyl or hexyl, or
a pharmaceutically acceptable salt or ester thereof,
so as to thereby reduce the toxicity of amyloidosis from islet amyloid polypeptide (IAPP) in the patient.

3. The method of claim 2, wherein the toxicity is to islet cells, β-cells or insulin-producing cells.

4. (canceled)

5. (canceled)

6. The method of claim 2, wherein the toxicity causes β-cell dysfunction, β-cell cell death, decline in β-cell mass, insulin resistance, impaired glucose tolerance or insulin deficiency in the patient.

7. (canceled)

8. (canceled)

9. A method of enhancing the survival of islet cell transplants in a patient, comprising administering to the patient a compound having the structure: 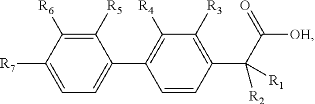
wherein R1 and R2 are each independently hydrogen, methyl, ethyl, propyl, butyl, pentyl or hexyl, or R1 and R2 are linked so as to form a cyclopropyl, cyclobutyl, cyclopentyl, or cyclohexyl ring;
wherein R3, R4, R5, R6 are each independently hydrogen, fluorine or chlorine; and
wherein R7 is hydrogen, phenyl, fluorine, chlorine, bromine, hydroxyl, amine, carboxylic acid, C1-C6 alkyl carboxylate, amide, methyl, ethyl, propyl, butyl, pentyl or hexyl, or
a pharmaceutically acceptable salt or ester thereof,
so as to thereby enhance the survival of islet cell transplants.

10. The method of claim 9, wherein the compound is administered to the patient before the patient receives islet cell transplants.

11. The method of claim 9, wherein the compound is administered to the patient after the patient receives islet cell transplants.

12. The method of claim 9, wherein the compound is administered to the patient concurrently with islet cell transplants.

13. (canceled)

14. The method of claim 9, wherein the islet cell transplants are human islet cells.

15. The method according to claim 1, wherein the compound has the structure: 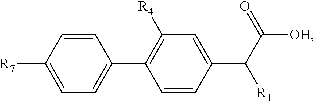
wherein
R1 is hydrogen or methyl;
R4 hydrogen or fluorine; and
R7 is hydrogen or hydroxyl, or
a pharmaceutically acceptable salt or ester thereof.

16. The method according to claim 1, wherein the compound has the structure: 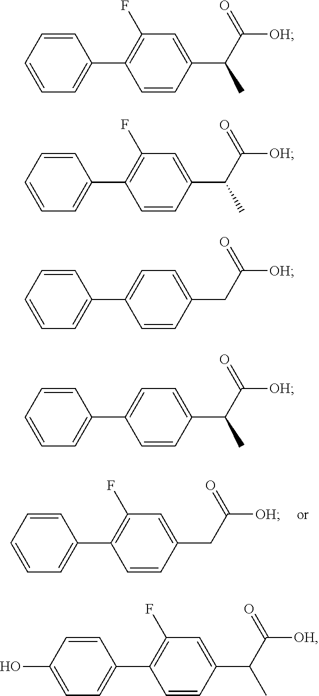
or
a pharmaceutically acceptable salt or ester thereof.

17. The method according to claim 1, wherein the compound has the structure: 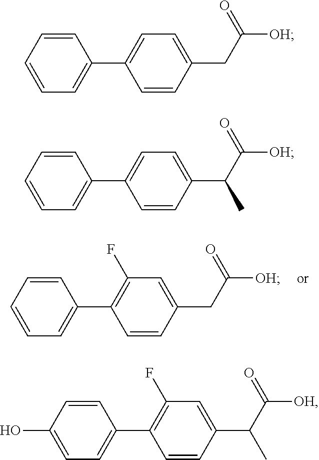
or
a pharmaceutically acceptable salt or ester thereof.

18. The method according to claim 1, wherein the compound has the structure: 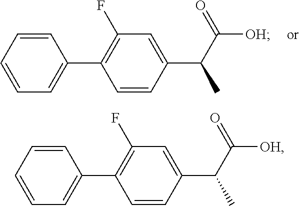
or
a pharmaceutically acceptable salt or ester thereof.

19. (canceled)

20. (canceled)

21. (canceled)

22. (canceled)

25. The method according to claim 1, wherein the amyloidosis from islet amyloid polypeptide (IAPP) is in the pancreas or in the islet of Langerhans.

26. (canceled)

27. The method according to claim 1, wherein the patient is a human.

28. The method according to claim 27, wherein the human exhibits a mutant form of islet amyloid polypeptide (IAPP).

29. The method according claim 1, wherein the patient is a domesticated animal, or is a cat or ferret.

30. (canceled)

31. (canceled)

32. The method according to claim 1, wherein the compound is administered orally, intravenously, intranasally, transdermally, intraperitoneal, subcutaneously, intramuscularly or via direct injection to the pancreas.

33. (canceled)

34. (canceled)

35. The method according to claim 1, wherein the compound is administered with a pharmaceutically acceptable carrier.

36. The method according to claim 1, wherein the compound is co-administered with insulin.
